(19) United States
(12) Patent Application Publication
(10) Pub. No.: US 2022/0062349 A1
(43) Pub. Date: Mar. 3, 2022

(54) DECELLULARIZATION AND RECELLULARIZATION OF ORGANS AND TISSUES

(71) Applicant: Miromatrix Medical Inc., Eden Prairie, MN (US)

(72) Inventors: Doris Taylor, St. Paul, MN (US); Harald Ott, Wenham, MA (US)

(21) Appl. No.: 17/394,243

(22) Filed: Aug. 4, 2021

Related U.S. Application Data
(63) Continuation of application No. 13/262,286, filed on Apr. 2, 2012, now abandoned.
(60) Provisional application No. 61/211,613, filed on Mar. 31, 2009.

(51) Int. Cl. A61K 35/55; C12M 1/12; A61L 27/38; C12M 1/00; A61L 27/36; C12M 3/00; A61L 27/54; A61K 35/407

(52) U.S. Cl. CPC A61K 35/55; C12M 25/14; A61L 27/3804; C12M 29/10; A61L 27/3604; C12M 21/08; A61L 2300/426; A61K 35/407; A61L 27/3839; A61L 2300/42; A61L 2300/414; A61L 2300/43; A61L 27/54

(57) ABSTRACT

The invention provides for methods and materials to decellularize an organ or portion thereof and to recellularize such a decellularized organ or portion thereof to thereby generate an organ or portion thereof.

### TECHNICAL FIELD

This invention relates to organs and tissues, and more particularly to methods and materials for decellularizing and recellularizing organs and tissues.

### BACKGROUND

Biologically derived matrices have been developed for tissue engineering and regeneration. The matrices developed to date, however, generally have a compromised matrix structure and/or do not exhibit a vascular bed that allows for effective reconstitution of the organ or tissue. This disclosure describes methods for decellularization and recellularization of organs and tissues.

### SUMMARY

This disclosure provides for methods and materials to decellularize an organ or tissue as well as methods and materials to recellularize a decellularized organ or tissue.

In one aspect, a decellularized mammalian heart is provided. A decellularized mammalian heart includes a decellularized extracellular matrix of the heart that has an exterior surface. The extracellular matrix of a decellularized heart substantially retains the morphology of the extracellular matrix prior to decellularization, and the exterior surface of the extracellular matrix is substantially intact.

Representative hearts include but are not limited to rodent hearts, pig hearts, rabbit hearts, bovine hearts, sheep hearts, or canine hearts. Another representative heart is a human heart. The decellularized heart can be cadaveric. In some embodiment, the decellularized heart is a portion of an entire heart. For example, a portion of an entire heart can include, without limitation, a cardiac patch, an aortic valve, a mitral valve, a pulmonary valve, a tricuspid valve, a right atrium, a left atrium, a right ventricle, a left ventricle, septum, coronary vasculature, a pulmonary artery, or a pulmonary vein.

In another aspect, a solid organ is provided. A solid organ as described herein includes the decellularized heart described above and a population of regenerative cells attached thereto. In some embodiments, the regenerative cells are pluripotent cells. In some embodiment, the regenerative cells are embryonic stem cells, umbilical cord cells, adult-derived stem or progenitor cells, bone marrow-derived cells, blood-derived cells, mesenchymal stem cells (MSC), skeletal muscle-derived cells, multipotent adult progenitor cells (MAPC), cardiac stem cells (CSC), or multipotent adult cardiac-derived stem cells. In some embodiments, the regenerative cells are cardiac fibroblasts, cardiac microvasculature cells, or aortic endothelial cells. In some embodiments, the cells are tissue-derived or skin-derived cells.

Generally, the number of the regenerative cells attached to the decellularized heart is at least about 1,000. In some embodiments, the number of the regenerative cells attached to the decellularized heart is about 1,000 cells/mg tissue (wet weight; i.e., pre-decellularized weight) to about 10,000,000 cells/mg tissue (wet weight). In some embodiments, the regenerative cells are heterologous to the decellularized heart. Also in some embodiments, the solid organ is to be transplanted into a patient and the regenerative cells are autologous to the patient.

In yet another aspect, a method of making a solid organ is provided. Such a method generally includes providing a decellularized heart as described herein, and contacting the decellularized heart with a population of regenerative cells under conditions in which the regenerative cells engraft, multiply and/or differentiate within and on the decellularized heart. In one embodiment, the regenerative cells are injected or perfused into the decellularized heart.

In still another aspect, a method of decellularizing a heart is provided. Such a method includes providing a heart, cannulating the heart at one or more than one cavity, vessel, and/or duct to produce a cannulated heart, and perfusing the cannulated heart with a first cellular disruption medium via the one or more than one cannulations. For example, the perfusion can be multi-directional from each cannulated cavity, vessel, and/or duct. Typically, the cellular disruption medium comprises at least one detergent such as SDS, PEG, or Triton X.

Such a method also can include perfusing the cannulated heart with a second cellular disruption medium via the more than one cannulations. Generally, the first cellular disruption medium can be an anionic detergent such as SDS and the second cellular disruption medium can be an ionic detergent such as Triton X-100. In such methods, the perfusing can be for about 2 to 12 hours per gram (wet weight) of heart tissue.

In one aspect, a solid organ is provided. Such a solid organ includes a decellularized organ and a population of regenerative cells attached thereto. Such a decellularized organ comprises a decellularized extracellular matrix of the organ, wherein the extracellular matrix comprises an exterior surface, and wherein the extracellular matrix, including the vascular tree, substantially retains the morphology of the extracellular matrix prior to decellularization, and wherein the exterior surface is substantially intact.

Representative solid organs include a heart, a kidney, a liver, or a lung. In one embodiment, the solid organ is a liver or a portion of a liver. In another embodiment, the solid organ is a heart (e.g., a rodent heart, a pig heart, a rabbit heart, a bovine heart, a sheep heart, or a canine heart; e.g., a heart that exhibits contractile activity). A representative heart is a human heart. The heart can be a portion of an entire heart (e.g., an aortic valve, a mitral valve, a pulmonary valve, a tricuspid valve, a right atrium, a left atrium, a right ventricle, a left ventricle, a cardiac patch, septum, a coronary vessel, a pulmonary artery, and a pulmonary vein). In another embodiment, the solid organ is a kidney. The solid organs described herein typically include multiple histological structures including blood vessels.

In some embodiments, the number of the regenerative cells attached to the decellularized organ is at least about 1,000. In other embodiments, the number of the regenerative cells attached to the decellularized organ is about 1,000 cells/mg tissue to about 10,000,000 cells/mg tissue. Regenerative cells can be pluripotent cells. Alternatively, the regenerative cells can be embryonic stem cells or a subset thereof, umbilical cord cells or a subset thereof, bone marrow cells or a subset thereof, peripheral blood cells or a subset thereof, adult-derived stem or progenitor cells or a subset thereof, tissue-derived stem or progenitor cells or a subset thereof, mesenchymal stem cells (MSC) or a subset thereof, skeletal muscle-derived stem or progenitor cells or a subset thereof, multipotent adult progenitor cells (MAPC) or a subset thereof, cardiac stem cells (CSC) or a subset thereof, or multipotent adult cardiac-derived stem cells or a subset thereof. Examples of regenerative cells include cardiac fibroblasts, cardiac microvasculature endothelial cells, aortic endothelial cells, or hepatocytes. In some embodiments, the regenerative cells are allogeneic or xenogeneic to the decellularized organ.

In some embodiments, the solid organ is to be transplanted into a patient and the regenerative cells are autologous to the patient. In other embodiments, the solid organ is to be transplanted into a patient and the decellularized organ is allogeneic or xenogeneic to the patient.

In another aspect, a method of making an organ is provided. Such methods generally include providing a decellularized organ, wherein the decellularized organ comprises a decellularized extracellular matrix of the organ, wherein the extracellular matrix comprises an exterior surface, and wherein the extracellular matrix, including the vascular tree, substantially retains the morphology of the extracellular matrix prior to decellularization, and wherein the exterior surface is substantially intact; and contacting the decellularized organ with a population of regenerative cells under conditions in which the regenerative cells engraft, multiply and/or differentiate within and on the decellularized organ. In one embodiment, the regenerative cells are injected into the decellularized organ. Representative decellularized organs include a heart, a kidney, a liver, spleen, pancreas, or a lung.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is a schematic showing the initial preparation for the decellularization of a heart. The aorta, pulmonary artery, and superior caval vein are cannulated (A, B, C, respectively), and the inferior caval vein, brachiocephalic artery, left common carotid artery, and left subclavian artery are ligated. Arrows indicate the direction of perfusion in antegrade and retrograde.

FIG. 2 is a schematic of one embodiment of a decellularization/recellularization apparatus.

FIG. 3A are photographs of liver and kidney being decellularized and FIG. 3B are photographs of heart and lung being decellularized. Photographs on the left show histology staining of tissue and describe the quantification of nucleic acid remaining in cadaveric organs, while the photographs on the right show histology staining of the decellularized matrix and quantification of nucleic acid remaining in perfusion-decellularized organs.

FIG. 4 show photographs of a perfusion decellularized pig kidney (left) and rat kidney (center; insets showing perfusion with Evans blue dye) and EM photographs of the glomerulus surrounded by tubules and the collecting ducts following perfusion decellularization.

FIG. 5 is a photograph of an entire rat decellularized from the lower abdomen to the head.

FIG. 6 are photographs showing recellularization of liver. FIG. 6A shows a perfusion decellularized rat liver; and FIG. 6B shows the injection of primary hepatocytes into a single lobe of a decellularized rat liver via a portal vein catheter.

FIG. 7 are photographs showing that recellularization can be targeted. FIG. 7A shows (primary rat hepatocytes being delivered to the caudate lobe of a decellularized liver; and FIG. 7B shows primary rat hepatocytes being delivered to the inferior/superior right lateral lobes of a decellularized rat liver.

FIG. 8 are SEM photographs showing the recellularization of decellularized rat liver. 40 million primary rat hepatocytes were delivered via a portal vein and cultured for 1 week (A-D).

FIG. 9 shows staining of recellularized rat liver one week after injection of primary rat hepatocytes into the caudate process. FIG. 9A is Masson's Trichrome staining (10×) and FIG. 9B is H&E staining (10×).

FIG. 10 shows TUNEL analysis of liver one week after recellularization with primary rat hepatocytes into the caudate process. FIG. 10A is TUNEL staining showing a mix of live and apoptotic cells (10×) and FIG. 10B is Masson's Trichrome staining (10×).

FIG. 11 shows Masson's Trichrome staining of human HepG2 cells after 1 week in perfusion decellularized rat liver in vitro. FIG. 11A shows the caudate process and FIG. 11B shows the superior/inferior right lateral lobe. Both are at 10×; V=vessels in the matrix.

FIG. 12 is a graph of cell retention efficiency. The graph shows that primary rat hepatocytes (1-6) or HepG2 (7 and 8) cells are retained after injection. Cells were counted before and after injection. Percent retention was calculated based on initial number minus retained cells.

FIG. 13 is a graph showing that HepG2 cells remain viable in the decellularized organ. Alamar blue metabolism demonstrates that HepG2 cells (˜30 million on the day of injection) remained viable and proliferated to a limited extent after injection into the caudate process (diamond) and the superior/inferior right lateral lobe (square).

FIG. 14 is a graph showing a time course of urea production by primary rat hepatocytes after recellularization (˜35 million cells for 7 days).

FIG. 15 is a graph showing a time course of albumin production every day by primary rat hepatocytes after recellularization (˜35 million cells for 7 days).

FIG. 16 is a graph showing a time course of ethoxyresorufin-O-deethylase (EROD) activity from primary rat hepatocytes injected in the caudate lobe (2.3 million cells for 8 days).

FIG. 17 is graphs showing that embryonic and adult-derived stem/progenitor cells proliferated for at least 3 weeks on decellularized heart, lung, liver, and kidney.

FIG. 18 is a graph showing that mouse embryonic stem cells (mESC) and proliferating adult stem cells (skeletal myoblasts; SKMB) were viable on decellularized heart, lung, liver, and kidney.

FIG. 19 are SEM photos of cadaveric (left panels) and decellularized (right panels) heart. LV, left ventricle; RV, right ventricle.

FIG. 20 are histological (top panels) and SEM (bottom panels) comparisons of cadaveric (left panels) and recellularized rat liver (right panels).

FIG. 21 is a photograph showing (A) a fully decellularized pig liver matrix, and SEM of perfusion decellularized pig liver showing (B) vascular conduits and (C) parenchymal matrix integrity.

FIG. 22 are photographs showing a gross view of immersion decellularized liver. Despite a gross appearance of intact liver, fraying of the matrix and a loss of capsule can be seen at both low (A) and higher (B) magnification.

FIG. 23 are SEM photographs showing that, after immersion decellularization (A and B), the organs lacked the Glisson's Capsule, while after 1% SDS perfusion decellularization (C and D), the organs retained the capsule.

FIG. 24 are photographs showing the histology of immersion decellularized rat liver (A, H&E; B, Trichrome) and the histology after 1% SDS perfusion decellularization (C, H&E; D, Trichrome).

FIG. 25 are photographs that show a comparison between immersion decellularization (top row) and perfusion decellularization (bottom row) of a rat heart. Left column, whole organ; Middle column, H&E tissue staining; Right column, SEM.

FIG. 26 are photographs that show a comparison between immersion decellularization (top row) and perfusion decellularization (bottom row) using rat kidney. Left column, whole organ; Middle column, H&E tissue staining; Right column, SEM.

FIG. 27 are SEM photographs of perfusion-decellularized kidney (FIG. 27A) and immersion-decellularized kidney (FIG. 27B).

FIG. 28 are SEM photographs of perfusion-decellularized heart (FIG. 28A) and immersion-decellularized heart (FIG. 28B).

FIG. 29 are SEM photographs of immersion-decellularized liver.

Like reference symbols in the various drawings indicate like elements.

### DETAILED DESCRIPTION

Solid organs generally have three main components, the extracellular matrix (ECM), cells embedded therein, and a vasculature bed. Decellularization of a solid organ as described herein removes most or all of the cellular components while substantially preserving the extracellular matrix (ECM) and the vasculature bed. A decellularized solid organ then can be used as a scaffold for recellularization. Mammals from which solid organs can be obtained include, without limitation, rodents, pigs, rabbits, cattle, sheep, dogs, and humans. Organs and tissues used in the methods described herein can be cadaveric, or can be fetal, neonatal, or adult.

Solid organs as referred to herein include, without limitation, heart, liver, lungs, skeletal muscles, brain, pancreas, spleen, kidneys, stomach, uterus, and bladder. A solid organ as used herein refers to an organ that has a “substantially closed” vasculature system. A “substantially closed” vasculature system with respect to an organ means that, upon perfusion with a liquid, the majority of the liquid is contained within the solid organ and does not leak out of the solid organ, assuming the major vessels are cannulated, ligated, or otherwise restricted. Despite having a “substantially closed” vasculature system, many of the solid organs listed above have defined “entrance” and “exit” vessels which are useful for introducing and moving the liquid throughout the organ during perfusion.

In addition to the solid organs described above, other types of vascularized organs or tissues such as, for example, all or portions of joints (e.g., knees, shoulders, hips or vertebrae), trachea, skin, mesentery or gut, small and large bowel, esophagus, ovaries, penis, testes, spinal cord, or single or branched vessels can be decellularized using the methods disclosed herein. Further, the methods disclosed herein also can be used to decellularize avascular (or relatively avascular) tissues such as, for example, cartilage or cornea.

A decellularized organ or tissue as described herein (e.g., heart or liver) or any portion thereof (e.g., an aortic valve, a mitral valve, a pulmonary valve, a tricuspid valve, a pulmonary vein, a pulmonary artery, coronary vasculature, septum, a right atrium, a left atrium, a right ventricle, a left ventricle or a hepatic lobe), with or without recellularization, can be used for transplanting into a patient. Alternatively, a recellularized organ or tissue as described herein can be used to examine, for example, cells undergoing differentiation and/or the cellular organization of an organ or tissue.

### Decellularization of Organs or Tissues

The invention provides for methods and materials to decellularize mammalian organ or tissue. The initial step in decellularizing an organ or tissue is to cannulate the organ or tissue, if possible. The vessels, ducts, and/or cavities of an organ or tissue can be cannulated using methods and materials known in the art. The next step in decellularizing an organ or tissue is to perfuse the cannulated organ or tissue with a cellular disruption medium. Perfusion through an organ can be multi-directional (e.g., antegrade and retrograde).

Langendorff perfusion of a heart is routine in the art, as is physiological perfusion (also known as four chamber working mode perfusion). See, for example, Dehnert, The Isolated Perfused Warm-Blooded Heart According to Langendorff, In Methods in Experimental Physiology and Pharmacology: Biological Measurement Techniques V. Biomesstechnik-Verlag March GmbH, West Germany, 1988. Briefly, for Langendorff perfusion, the aorta is cannulated and attached to a reservoir containing cellular disruption medium. A cellular disruption medium can be delivered in a retrograde direction down the aorta either at a constant flow rate delivered, for example, by an infusion or roller pump or by a constant hydrostatic pressure. In both instances, the aortic valves are forced shut and the perfusion fluid is directed into the coronary ostia (thereby perfusing the entire ventricular mass of the heart), which then drains into the right atrium via the coronary sinus. For working mode perfusion, a second cannula is connected to the left atrium and perfusion can be changed from retrograde to antegrade.

Methods are known in the art for perfusing other organ or tissues. By way of example, the following references describe the perfusion of lung, liver, kidney, brain, and limbs. Van Putte et al., 2002, Ann. Thorac. Surg., 74(3):893-8; den Butter et al., 1995, Transpl. Int., 8:466-71; Firth et al., 1989, Clin. Sci. (Lond.), 77(6):657-61; Mazzetti et al., 2004, Brain Res., 999(1):81-90; Wagner et al., 2003, J. Artif. Organs, 6(3):183-91.

One or more cellular disruption media can be used to decellularize an organ or tissue. A cellular disruption medium generally includes at least one detergent such as SDS, PEG, or Triton X. A cellular disruption medium can include water such that the medium is osmotically incompatible with the cells, Alternatively, a cellular disruption medium can include a buffer (e.g., PBS) for osmotic compatibility with the cells. Cellular disruption media also can include enzymes such as, without limitation, one or more collagenases, one or more disposes, one or more DNases, or a protease such as trypsin. In some instances, cellular disruption media also or alternatively can include inhibitors of one or more enzymes (e.g., protease inhibitors, nuclease inhibitors, and/or collegenase inhibitors).

In certain embodiments, a cannulated organ or tissue can be perfused sequentially with two different cellular disruption media. For example, the first cellular disruption medium can include an anionic detergent such as SDS and the second cellular disruption medium can include an ionic detergent such as Triton X-100. Following perfusion with at least one cellular disruption medium, a cannulated organ or tissue can be perfused, for example, with wash solutions and/or solutions containing one or more enzymes such as those disclosed herein.

Alternating the direction of perfusion (e.g., antegrade and retrograde) can help to effectively decellularize the entire organ or tissue. Decellularization as described herein essentially decellularizes the organ from the inside out, resulting in very little damage to the ECM. An organ or tissue can be decellularized at a suitable temperature between 4 and 40° C. Depending upon the size and weight of an organ or tissue and the particular detergent(s) and concentration of detergent(s) in the cellular disruption medium, an organ or tissue generally is perfused from about 2 to about 12 hours per gram of solid organ or tissue with cellular disruption medium. Including washes, an organ may be perfused for up to about 12 to about 72 hours per gram of tissue. Perfusion generally is adjusted to physiologic conditions including pulsatile flow, rate and pressure.

As indicated herein, a decellularized organ or tissue consists essentially of the extracellular matrix (ECM) component of all or most regions of the organ or tissue, including ECM components of the vascular tree. ECM components can include any or all of the following: fibronectin, fibrillin, elastin, members of the collagen family (e.g., collagen I, III, and IV), glycosaminoglycans, ground substance, reticular fibers and thrombospondin, which can remain organized as defined structures such as the basal lamina. Successful decellularization is defined as the absence of detectable myofilaments, endothelial cells, smooth muscle cells, and nuclei in histologic sections using standard histological staining procedures. Preferably, but not necessarily, residual cell debris also has been removed from the decellularized organ or tissue.

To effectively recellularize and generate an organ or tissue, it is important that the morphology and the architecture of the ECM be maintained (i.e., remain substantially intact) during and following the process of decellularization. “Morphology” as used herein refers to the overall shape of the organ or tissue or of the ECM, while “architecture” as used herein refers to the exterior surface, the interior surface, and the ECM therebetween.

The morphology and architecture of the ECM can be examined visually and/or histologically. For example, the basal lamina on the exterior surface of a solid organ or within the vasculature of an organ or tissue should not be removed or significantly damaged due to decellularization. In addition, the fibrils of the ECM should be similar to or significantly unchanged from that of an organ or tissue that has not been decellularized. Unless indicated otherwise, decellularization as used herein refers to perfusion decellularization and, unless indicated otherwise, a decellularized organ or matrix referred to herein is obtained using the perfusion decellularization described herein. Perfusion decellularization as described herein can be compared to immersion decellularization as described, for example, in U.S. Pat. Nos. 6,753,181 and 6,376,244.

One or more compounds can be applied in or on a decellularized organ or tissue to, for example, preserve the decellularized organ, or to prepare the decellularized organ or tissue for recellularization and/or to assist or stimulate cells during the recellularization process. Such compounds include, but are not limited to, one or more growth factors (e.g., VEGF, DKK-1, FGF, BMP-1, BMP-4, SDF-1, IGF, and HGF), immune modulating agents (e.g., cytokines, glucocorticoids, IL2R antagonist, leucotriene antagonists), and/or factors that modify the coagulation cascade (e.g., aspirin, heparin-binding proteins, and heparin). In addition, a decellularized organ or tissue can be further treated with, for example, irradiation (e.g., UV, gamma) to reduce or eliminate the presence of any type of microorganism remaining on or in a decellularized organ or tissue.

### Recellularization of Organs or Tissues

The invention provides for materials and methods for generating an organ or tissue. An organ or tissue can be generated by contacting a decellularized organ or tissue as described herein with a population of regenerative cells. Regenerative cells as used herein are any cells used to recellularize a decellularized organ or tissue. Regenerative cells can be totipotent cells, pluripotent cells, or multipotent cells, and can be uncommitted or committed. Regenerative cells also can be single-lineage cells. In addition, regenerative cells can be undifferentiated cells, partially differentiated cells, or fully differentiated cells. Regenerative cells as used herein include embryonic stem cells (as defined by the National Institute of Health (NIH); see, for example, the Glossary at stemcells.nih.gov on the World Wide Web). Regenerative cells also include progenitor cells, precursor cells, and “adult”-derived stem cells including umbilical cord cells and fetal stem cells.

Examples of regenerative cells that can be used to recellularize an organ or tissue include, without limitation, embryonic stem cells, umbilical cord blood cells, tissue-derived stem or progenitor cells, bone marrow-derived stem or progenitor cells, blood-derived stem or progenitor cells, adipose tissue-derived stem or progenitor cells, mesenchymal stem cells (MSC), skeletal muscle-derived cells, or multipotent adult progenitor cells (MAPC). Additional regenerative cells that can be used include tissue-specific stem cells including cardiac stem cells (CSC), multipotent adult cardiac-derived stem cells, cardiac fibroblasts, cardiac microvasculature endothelial cells, or aortic endothelial cells. Bone marrow-derived stem cells such as bone marrow mononuclear cells (BM-MNC), endothelial or vascular stem or progenitor cells, and peripheral blood-derived stem cells such as endothelial progenitor cells (EPC) also can be used as regenerative cells.

The number of regenerative cells that is introduced into and onto a decellularized organ in order to generate an organ or tissue is dependent on both the organ (e.g., which organ, the size and weight of the organ) or tissue and the type and developmental stage of the regenerative cells. Different types of cells may have different tendencies as to the population density those cells will reach. Similarly, different organ or tissues may be recellularized at different densities. By way of example, a decellularized organ or tissue can be “seeded” with at least about 1,000 (e.g., at least 10,000, 100,000, 1,000,000, 10,000,000, or 100,000,000) regenerative cells; or can have from about 1,000 cells/mg tissue (wet weight, i.e., prior to decellularization) to about 10,000,000 cells/mg tissue (wet weight) attached thereto.

Regenerative cells can be introduced (“seeded”) into a decellularized organ or tissue by injection into one or more locations. In addition, more than one type of cell (i.e., a cocktail of cells) can be introduced into a decellularized organ or tissue. For example, a cocktail of cells can be injected at multiple positions in a decellularized organ or tissue or different cell types can be injected into different portions of a decellularized organ or tissue. Alternatively, or in addition to injection, regenerative cells or a cocktail of cells can be introduced by perfusion into a cannulated decellularized organ or tissue. For example, regenerative cells can be perfused into a decellularized organ using a perfusion medium, which can then be changed to an expansion and/or differentiation medium to induce growth and/or differentiation of the regenerative cells.

During recellularization, an organ or tissue is maintained under conditions in which at least some of the regenerative cells can multiply and/or differentiate within and on the decellularized organ or tissue. Those conditions include, without limitation, the appropriate temperature and/or pressure, electrical and/or mechanical activity, force, the appropriate amounts of O₂ and/or CO₂, an appropriate amount of humidity, and sterile or near-sterile conditions. During recellularization, the decellularized organ or tissue and the regenerative cells attached thereto are maintained in a suitable environment. For example, the regenerative cells may require a nutritional supplement (e.g., nutrients and/or a carbon source such as glucose), exogenous hormones or growth factors, and/or a particular pH.

Regenerative cells can be allogeneic to a decellularized organ or tissue (e.g., a human decellularized organ or tissue seeded with human regenerative cells), or regenerative cells can be xenogeneic to a decellularized organ or tissue (e.g., a pig decellularized organ or tissue seeded with human regenerative cells). “Allogeneic” as used herein refers to cells obtained from the same species as that from which the organ or tissue originated (e.g., self (i.e., autologous) or related or unrelated individuals), while “xenogeneic” as used herein refers to cells obtained from a species different than that from which the organ or tissue originated.

In some instances, an organ or tissue generated by the methods described herein is to be transplanted into a patient. In those cases, the regenerative cells used to recellularize a decellularized organ or tissue can be obtained from the patient such that the regenerative cells are “autologous” to the patient. Regenerative cells from a patient can be obtained from, for example, blood, bone marrow, tissues, or organs at different stages of life (e.g., prenatally, neonatally or perinatally, during adolescence, or as an adult) using methods known in the art. Alternatively, regenerative cells used to recellularize a decellularized organ or tissue can be syngeneic (i.e., from an identical twin) to the patient, regenerative cells can be human lymphocyte antigen (HLA)-matched cells from, for example, a relative of the patient or an HLA-matched individual unrelated to the patient, or regenerative cells can be allogeneic to the patient from, for example, a non-HLA-matched donor.

Irrespective of the source of the regenerative cells (e.g., autologous or not), the decellularized solid organ can be autologous, allogeneic or xenogeneic to a patient.

In certain instances, a decellularized organ may be recellularized with cells in vivo (e.g., after the organ or tissue has been transplanted into an individual). In vivo recellularization may be performed as described above (e.g., injection and/or perfusion) with, for example, any of the regenerative cells described herein. Alternatively or additionally, in vivo seeding of a decellularized organ or tissue with endogenous cells may occur naturally or be mediated by factors delivered to the recellularized tissue.

The progress of regenerative cells can be monitored during recellularization. For example, the number of cells on or in an organ or tissue can be evaluated by taking a biopsy at one or more time points during recellularization. In addition, the amount of differentiation that regenerative cells have undergone can be monitored by determining whether or not various markers are present in a cell or a population of cells. Markers associated with different cells types and different stages of differentiation for those cell types are known in the art, and can be readily detected using antibodies and standard immunoassays. See, for example, Current Protocols in Immunology, 2005, Coligan et al., Eds., John Wiley Sons, Chapters 3 and 11. Nucleic acid assays as well as morphological and/or histological evaluation can be used to monitor recellularization. Functional analysis of recellularized organs also can be evaluated. For example, contractions and ventricular pressure can be evaluated in a recellularized heart; albumin production, urea production, and cytochrome p450 activity can be evaluated in a recellularized liver; blood or media filtration and urine production can be evaluated in a recellularized kidney; blood, glucose and insulin can be evaluated in a recellularized pancreas; force generation or response to stimulation can be evaluated in a recellularized muscle; and thrombogenicity can be evaluated in a recellularized vessel.

Controlled System for Decellularizing and/or Recellularizing an Organ or Tissue

The invention also provides for a system (e.g., a bioreactor) for decellularizing and/or recellularizing an organ or tissue. Such a system generally includes at least one cannulation device for cannulating an organ or tissue, a perfusion apparatus for perfusing the organ or tissue through the cannula(s), and means (e.g., a containment system) to maintain a sterile environment for the organ or tissue. Cannulation and perfusion are well-known techniques in the art. A cannulation device generally includes size-appropriate hollow tubing for introducing into a vessel, duct, and/or cavity of an organ or tissue. Typically, one or more vessels, ducts, and/or cavities are cannulated in an organ. A perfusion apparatus can include a holding container for the liquid (e.g., a cellular disruption medium) and a mechanism for moving the liquid through the organ (e.g., a pump, air pressure, gravity) via the one or more cannulae. The sterility of an organ or tissue during decellularization and/or recellularization can be maintained using a variety of techniques known in the art such as controlling and filtering the air flow and/or perfusing with, for example, antibiotics, anti-fungals or other anti-microbials to prevent the growth of unwanted microorganisms.

A system to decellularize and recellularize organ or tissues as described herein can possess the ability to monitor certain perfusion characteristics (e.g., pressure, volume, flow pattern, temperature, gases, pH), mechanical forces (e.g., ventricular wall motion and stress), and electrical stimulation (e.g., pacing). As the coronary vascular bed changes over the course of decellularization and recellularization (e.g. vascular resistance, volume), a pressure-regulated perfusion apparatus is advantageous to avoid large fluctuations. The effectiveness of perfusion can be evaluated in the effluent and in tissue sections. Perfusion volume, flow pattern, temperature, partial O₂ and CO₂ pressures and pH can be monitored using standard methods.

Sensors can be used to monitor the system (e.g., bioreactor) and/or the organ or tissue. Sonomicromentry, micromanometry, and/or conductance measurements can be used to acquire pressure-volume or preload recruitable stroke work information relative to myocardial wall motion and performance. For example, sensors can be used to monitor the pressure of a liquid moving through a cannulated organ or tissue; the ambient temperature in the system and/or the temperature of the organ or tissue; the pH and/or the rate of flow of a liquid moving through the cannulated organ or tissue; and/or the biological activity of a recellularizing organ or tissue. In addition to having sensors for monitoring such features, a system for decellularizing and/or recellularizing an organ or tissue also can include means for maintaining or adjusting such features. Means for maintaining or adjusting such features can include components such as a thermometer, a thermostat, electrodes, pressure sensors, overflow valves, valves for changing the rate of flow of a liquid, valves for opening and closing fluid connections to solutions used for changing the pH of a solution, a balloon, an external pacemaker, and/or a compliance chamber. To help ensure stable conditions (e.g., temperature), the chambers, reservoirs and tubings can be water-jacketed.

It can be advantageous during recellularization to place a mechanical load on the organ and the cells attached thereto. As an example, a balloon inserted into the left ventricle via the left atrium can be used to place mechanical stress on a heart. A piston pump that allows adjustment of volume and rate can be connected to the balloon to simulate left ventricular wall motion and stress. To monitor wall motion and stress, left ventricular wall motion and pressure can be measured using micromanometry, sonomicrometry, pressure-volume changes, or echocardiography. In some embodiments, an external pacemaker can be connected to a piston pump to provide synchronized stimulation with each deflation of the ventricular balloon (which is equivalent to the systole). Peripheral ECG can be recorded from the heart surface to allow for the adjustment of pacing voltage, the monitoring of de- and repolarization, and to provide a simplified surface map of the recellularizing or recellularized heart.

Mechanical ventricular distention can also be achieved by attaching a peristaltic pump to a cannula inserted into the left ventricle through the left atrium. Similar to the procedure described above involving a balloon, ventricular distention achieved by periodic fluid movement (e.g., pulsatile flow) through the cannula can be synchronized with electrical stimulation.

Using the methods and materials disclosed herein, a mammalian heart can be decellularized and recellularized and, when maintained under the appropriate conditions, a functional heart that undergoes contractile function and responds to pacing stimuli and/or pharmacologic agents can be generated. This recellularized functional heart can be transplanted into a mammal and function for a period of time.

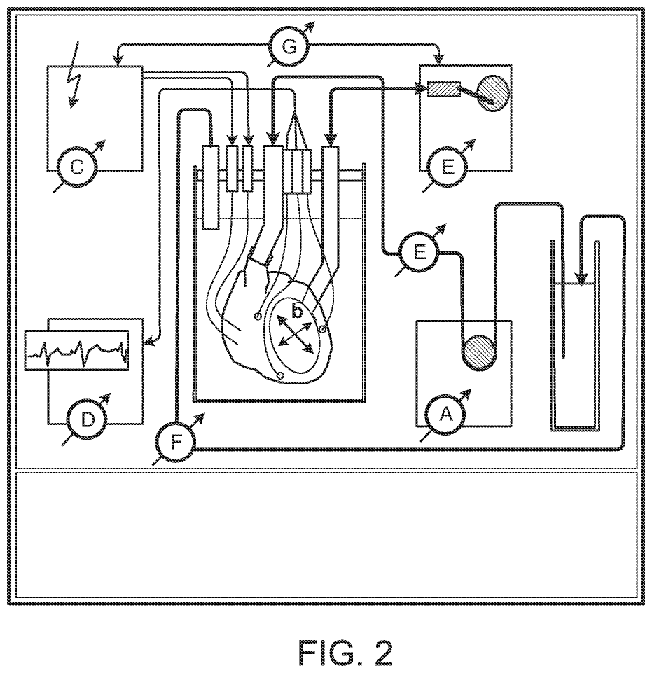

FIG. 2 shows one embodiment of a system for decellularizing and/or recellularizing an organ or tissue (e.g., a bioreactor). The embodiment shown is a bioreactor for decellularizing and recellularizing a heart. This embodiment has an adjustable rate and volume peristaltic pump (A); an adjustable rate and volume piston pump connected to an intraventricular balloon (B); an adjustable voltage, frequency and amplitude external pacemaker (C.); an ECG recorder (D); a pressure sensor in the ‘arterial line’ (which equals coronary artery pressure) (E); a pressure sensor in the ‘venous’ line (which equals coronary sinus pressure) (F); and synchronization between the pacemaker and the piston pump (G).

A system for generating an organ or tissue can be controlled by a computer-readable storage medium in combination with a programmable processor (e.g., a computer-readable storage medium as used herein has instructions stored thereon for causing a programmable processor to perform particular steps). For example, such a storage medium, in combination with a programmable processor, can receive and process information from one or more of the sensors. Such a storage medium in conjunction with a programmable processor also can transmit information and instructions back to the bioreactor and/or the organ or tissue.

An organ or tissue undergoing recellularization can be monitored for biological activity. The biological activity can be that of the organ or tissue itself such as electrical activity, mechanical activity, mechanical pressure, contractility, and/or wall stress of the organ or tissue. In addition, the biological activity of the cells attached to the organ or tissue can be monitored, for example, for ion transport/exchange activity, cell division, and/or cell viability. See, for example, Laboratory Textbook of Anatomy and Physiology (2001, Wood, Prentice Hall) and Current Protocols in Cell Biology (2001, Bonifacino et al., Eds, John Wiley & Sons). As discussed above, it may be useful to simulate an active load on an organ during recellularization. A computer-readable storage medium of the invention, in combination with a programmable processor, can be used to coordinate the components necessary to monitor and maintain an active load on an organ or tissue.

In one embodiment, the weight of an organ or tissue can be entered into a computer-readable storage medium as described herein, which, in combination with a programmable processor, can calculate exposure times and perfusion pressures for that particular organ or tissue. Such a storage medium can record preload and afterload (the pressure before and after perfusion, respectively) and the rate of flow. In this embodiment, for example, a computer-readable storage medium in combination with a programmable processor can adjust the perfusion pressure, the direction of perfusion, and/or the type of perfusion solution via one or more pumps and/or valve controls.

In accordance with the present invention, there may be employed conventional molecular biology, microbiology, biochemical, and cell biology techniques within the skill of the art. Such techniques are explained fully in the literature. The invention will be further described in the following examples, which do not limit the scope of the invention described in the claims.

### EXAMPLES

### Section A. Decellularization (Part 1)

### Example 1 Preparation of a Solid Organ for Decellularization

To avoid the formation of post mortal thrombi, a donor rat was systemically heparinized with 400 U of heparin/kg of donor. Following heparinization, the heart and the adjacent large vessels were carefully removed.

The heart was placed in a physiologic saline solution (0.9%) containing heparin (2000 U/ml) and held at 5° C. until further processing. Under sterile conditions, the connective tissue was removed from the heart and the large vessels. The interior vena cava and the left and right pulmonary veins were ligated distal from the right and left atrium using monofil, non-resorbable ligatures.

### Example 2—Cannulation and Perfusion of a Solid Organ

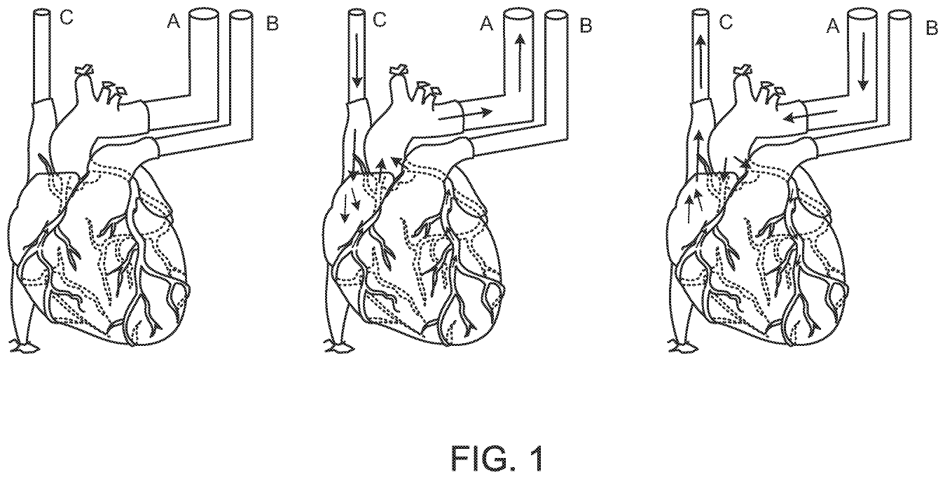

The heart was mounted on a decellularization apparatus for perfusion (FIG. 1). The descending thoracic artery was cannulated to allow retrograde coronary perfusion (FIG. 1, Cannula A). The branches of the thoracic artery (e.g., brachiocephalic trunc, left common carotid artery, left subclavian artery) were ligated. The pulmonary artery was cannulated before its division into the left and right pulmonary artery (FIG. 1, Cannula B). The superior vena cava was cannulated (FIG. 1, Cannula C). This configuration allows for both retrograde and antegrade coronary perfusion.

When positive pressure was applied to the aortic cannula (A), perfusion occurred from the coronary arteries through the capillary bed to the coronary venous system to the right atrium and the superior caval vein (C). When positive pressure was applied to the superior caval vein cannula (C), perfusion occurred from the right atrium, the coronary sinus, and the coronary veins through the capillary bed to the coronary arteries and the aortic cannula (A).

### Example 3—Decellularization

After the heart was mounted on the decellularization apparatus, antegrade perfusion was started with cold, heparinized, calcium-free phosphate buffered solution containing 1-5 mmol adenosine per L perfusate to reestablish constant coronary flow. Coronary flow was assessed by measuring the coronary perfusion pressure and the flow, and calculating coronary resistance. After 15 minutes of stable coronary flow, the detergent-based decellularization process was initiated.

The details of the procedures are described below. Briefly, however, a heart was perfused antegradely with a detergent. After perfusion, the heart can be flushed with a buffer (e.g., PBS) retrogradely. The heart then was perfused with PBS containing antibiotics and then PBS containing DNase I. The heart then was perfused with 1% benzalkonium chloride to reduce microbial contamination and to prevent future microbial contamination, and then perfused with PBS to wash the organ of any residual cellular components, enzymes, or detergent.

### Example 4—Decellularization of Cadaveric Rat Hearts

Hearts were isolated from 8 male nude rats (250-300 g). Immediately after dissection, the aortic arch was cannulated and the hearts were retrogradely perfused with the indicated detergent. The four different detergent-based decellularization protocols (see below) were compared with respect to their feasibility and efficacy in (a) removing cellular components and (b) preserving vascular structures.

Decellularization generally included the following steps: stabilization of the solid organ, decellularization of the solid organ, renaturation and/or neutralization of the solid organ, washing the solid organ, degradation of any DNA remaining on the organ, disinfection of the organ, and homeostasis of the organ.

A) Decellularization Protocol #1 (PEG)

Hearts were washed in 200 ml PBS containing 100 U/ml penicillin 0.1 mg/ml Streptomycin, and 0.25 μg/ml Amphotericin B with no recirculation. Hearts were then decellularized with 35 ml polyethyleneglycol (PEG; 1 g/ml) for up to 30 minutes with manual recirculation. The organ was then washed with 500 ml PBS for up to 24 hours using a pump for recirculation. The washing step was repeated at least twice for at least 24 hours each time. Hearts were exposed to 35 ml DNase I (70 U/ml) for at least 1 hour with manual recirculation. The organs were washed again with 500 ml PBS for at least 24 hours.

B) Decellularisation Protocol #2 (Triton X and Trypsin)

Hearts were washed in 200 ml PBS containing 100 U/ml Penicillin, 0.1 mg/ml Streptomycin, and 0.25 μg/ml Amphotericin B for at least about 20 minutes with no recirculation. Hearts were then decellularized with 0.05% Trypsin for 30 min followed by perfusion with 500 ml PBS containing 5% Triton-X and 0.1% ammonium-hydroxide for about 6 hours. Hearts were perfused with deionized water for about 1 hour, and then perfused with PBS for 12 h. Hearts were then washed 3 times for 24 hours each time in 500 ml PBS using a pump for recirculation. The hearts were perfused with 35 ml DNase I (70 U/ml) for 1 hour with manual recirculation and washed twice in 500 ml PBS for at least about 24 hours each time using a pump for recirculation.

Decellularization Protocol #3 (1% SDS)

Hearts were washed in 200 ml PBS containing 100 U/m1 Penicillin, 0.1 mg/ml Streptomycin, and 0.25 μg/ml Amphotericin B for at least about 20 mins with no recirculation. The hearts were decellularized with 500 ml water containing 1% SDS for at least about 6 hours using a pump for recirculation. The hearts were then washed with deionized water for about 1 hour and washed with PBS for about 12 hours. The hearts were washed three times with 500 ml PBS for at least about 24 hours each time using a pump for recirculation. The heart was then perfused with 35 ml DNase I (70 U/ml) for about 1 hour using manual recirculation, and washed three times with 500 ml PBS for at least about 24 hours each time using a pump for recirculation.

D) Decellularisation Protocol #4 (Triton X)

Hearts were washed with 200 ml PBS containing 100 U/ml Penicillin, 0.1 mg/ml Streptomycin, and 0.25 μg/ml Amphotericin B for at least about 20 mins with no recirculation. Hearts were then decellularized with 500 ml water containing 5%; Triton X and 0.1% ammonium hydroxide for at least 6 hours using a pump for recirculation. Hearts were then perfused with deionized water for about 1 hour and then with PBS for about 12 hours. Hearts were washed by perfusing with 500 ml PBS 3 times for at least 24 hours each time using a pump for recirculation. Hearts were then perfused with 35 ml DNase I (70 U/ml) for about 1 hour using manual recirculation, and washed three times in 500 ml PBS for about 24 hours each time.

For initial experiments, the decellularization apparatus was set up within a laminar flow hood. Hearts were perfused at a coronary perfusion pressure of 60 cm H₂O. Although not required, the hearts described in the experiments above were mounted in a decellularization chamber and completely submerged and perfused with PBS containing antibiotics for 72 hours in recirculation mode at a continuous flow of 5 ml/min to wash out as many cellular components and detergent as possible.

Successful decellularization was defined as the lack of myofilaments and nuclei in histologic sections. Successful preservation of vascular structures was assessed by perfusion with 2% Evans Blue prior to embedding tissue sections.

Highly efficient decellularization took place when a heart was first perfused antegradely with an ionic detergent (1% sodium-dodecyl-sulfate (SDS), approximately 0.03 M) dissolved in deionized H₂O at a constant coronary perfusion pressure and then was perfused antegradely with a non-ionic detergent (1% Triton X-100) to remove the SDS and presumably to renature the extracellular matrix (ECM) proteins. Intermittently, the heart was perfused retrogradely with phosphate buffered solution to clear obstructed capillaries and small vessels.

### Example 5—Evaluation of Decellularized Organs

To demonstrate intact vascular structures following decellularization, decellularized heart is stained via Langendorff perfusion with Evans Blue to stain vascular basement membrane and quantify macro- and micro-vascular density. Further, polystyrene particles can be perfused into and through a heart to quantify coronary volume, the level of vessel leakage, and to assess the distribution of perfusion by analyzing coronary effluent and tissue sections. A combination of three criteria are assessed and compared to isolated non-decellularised heart: 1) an even distribution of polystyrene particles, 2) significant change in leakiness at some level 3) microvascular density.

Fiber orientation is assessed by the polarized-light microscopy technique of Tower et al. (2002, Fiber alignment imaging during mechanical testing of soft tissues, Ann Biomed Eng., 30(10):1221-33), which can be applied in real-time to a sample subjected to uniaxial or biaxial stress. During Langendorff perfusion, basic mechanical properties of the decellularised ECM are recorded (compliance, elasticity, burst pressure) and compared to freshly isolated hearts.

### Section B. Decellularization (Part II)

### Example1—Decellularization of Rat Heart

Male 12 week old F344 Fischer rats (Harlan Labs, PO Box 29176 Indianapolis, Ind. 46229), were anesthetized using intraperitoneal injection of 100 mg/kg ketamine (Phoenix Pharmaceutical, Inc., St. Joseph, Mo.) and 10 mg/kg xylazine (Phoenix Pharmaceutical, Inc., St. Joseph, Mo.). After systemic heparinization (American Pharmaceutical Partners, Inc., Schaumberg, Ill.) through the left femoral vein, a median sternotomy was performed and the pericardium was opened. The retrosternal fat body was removed, the ascending thoracic aorta was dissected and its branches ligated. The caval and pulmonary veins, the pulmonary artery and the thoracic aorta were transected and the heart was removed from the chest. A prefilled 1.8 mm aortic cannula (Radnoti Glass, Monrovia, Calif.) was inserted into the ascending aorta to allow retrograde coronary perfusion (Langendorff). The hearts were perfused with heparinized PBS (Hyclone, Logan, Utah) containing 10 μM adenosine at a coronary perfusion pressure of 75 cm H₂O for 15 minutes followed by 1% sodium dodecyl sulfate (SDS) or 1% polyethylene glycol 1000 (PEG 1000) (EMD Biosciences, La Jolla, Germany) or 1% Triton-X 100 (Sigma, St. Louis, Mo.) in deionized water for 2-15 hours. This was followed by 15 minutes of deionized water perfusion and 30 minutes of perfusion with 1% Triton-X (Sigma, St. Louis, Mo.) in deionized water. The hearts were then continuously perfused with antibiotic-containing PBS (100 U/ml penicillin-G (Gibco, Carlsbad, Calif.), 100 U/ml streptomycin (Gibco, Carlsbad, Calif.) and 0.25 μg/ml Amphotericin B (Sigma, St. Louis, Mo.)) for 124 hours.

After 420 minutes of retrograde perfusion with either 1% PEG, 1% Triton-X 100 or 1% SDS, PEG and Triton-X 100 perfusion induced an edematous, opaque appearance, while SDS perfusion resulted in a more dramatic change leading to a nearly translucent graft as opaque elements were slowly washed out. Hearts exposed to all three protocols remained grossly intact with no evidence of coronary rupture or aortic valve insufficiency throughout the perfusion protocol (at constant coronary perfusion pressure of 77.4 mmHg), Coronary flow decreased in all three protocols during the first 60 minutes of perfusion, then normalized during SDS perfusion while remaining increased in Triton-X 100 and PEG perfusion. SDS perfusion induced the highest initial increase in calculated coronary resistance (up to 250 mmHg·s·ml⁻¹), followed by Triton-X (up to 200 mmHg·s·ml⁻¹) and PEG (up to 150 mmHg·s·ml⁻¹).

Using histological sections of the detergent perfused heart tissue, it was determined that decellularization over the observed time period was incomplete M both PEG and Triton-X 100 treated hearts; Hematoxylin-Eosin (H&E) staining showed nuclei and cross-striated filaments. In contrast, no nuclei or contractile filaments were detectable in sections of SDS-perfused hearts. Vascular structures and ECM fiber direction, however, were preserved in the SDS-treated hearts.

To remove the ionic SDS from the ECM after the initial decellularization, the organ was perfused for 30 minutes with Triton-X 100. In addition and to ensure complete washout of all detergents and to reestablish a physiologic pH, the decellularized organ was perfused extensively with deionized water and PBS for 124 h.

### Example 2—Decellularization of Rat Kidney

For kidney isolation, the entire peritoneal content was wrapped in wet gauze and carefully mobilized to the side to expose the retroperitoneal space. The mesenteric vessels were ligated and transected. The abdominal aorta was ligated and transected below the take off of the renal arteries. The thoracic aorta was transected just above the diaphragm and cannulated using a 1.8 mm aortic cannula (Radnoti Glass, Monrovia, Calif.). The kidneys were carefully removed from the retroperitoneum and submerged in sterile PBS (Hyclone, Logan, Utah) to minimize pulling force on the renal arteries. 15 minutes of heparinized PBS perfusion were followed by 2-16 hours of perfusion with 1% SDS (invitrogen, Carlsbad, Calif.) in deionized water and 30 minutes of perfusion with 1% Triton-X (Sigma, St. Louis, Mo.) in deionized water. The liver was then continuously perfused with antibiotic containing PBS (100 U/ml (Gibco, Carlsbad, Calif.), 100 U/ml streptomycin (Gibco, Carlsbad, Calif.), 0.25 μg/ml Amphotericin B (Sigma, St. Louis, Mo.)) for 124 hours.

420 minutes of SDS perfusion followed by Triton-X 100 yielded a completely decellularized renal ECM scaffold with intact vasculature and organ architecture. Evans blue perfusion confirmed intact vasculature similar to decellularized cardiac ECM. Movat pentachrome staining of decellularized renal cortex showed intact glomeruli and proximal and distal convoluted tubule basement membranes without any intact cells or nuclei. Staining of decellularized renal medulla showed intact tubule and collecting duct basement membranes. SEM of decellularized renal cortex confirmed intact glomerular and tubular basement membranes. Characteristic structures such as Bowman's capsule delineating the glomerulus from surrounding proximal and distal tubules and glomerular capillary basement membranes within the glomeruli were preserved. SEM images of decellularized renal medulla showed intact medullary pyramids reaching into the renal pelvis with intact collecting duct basal membranes leading towards the papilla. Thus, all the major ultrastructures of the kidney were intact after decellularization.

### Example 3—Decellularization of Rat Lung

The lung (with the trachea) were carefully removed from the chest and submerged in sterile PBS (Hyclone, Logan, Utah) to minimize pulling force on the pulmonary arteries. 15 minutes of heparinized PBS perfusion was followed by 2-12 hours of perfusion with 1% SDS (invitrogen, Carlsbad, Calif.) in deionized water and 15 minutes of perfusion with 1% Triton-X (Sigma, St. Louis, Mo.) in deionized water. The lung was then continuously perfused with antibiotic containing PBS (100 U/ml (Gibco, Carlsbad, Calif.), 100 U/ml streptomycin (Gibco, Carlsbad, Calif.), 0.25 μg/ml Amphotericin B (Sigma, St. Louis, Mo.)) for 124 hours.

180 minutes of SDS perfusion followed by Triton-X 100 perfusion yielded a completely decellularized pulmonary ECM scaffold with intact airways and vessels. Movat pentachrome staining of histologic sections showed the presence of ECM components in lung including major structural proteins such as collagen and elastin and also soluble elements such as proteoglycans. However, no nuclei or intact cells were retained. Airways were preserved from the main bronchus to terminal bronchiole to respiratory bronchioles, alveolar ducts and alveoles. The vascular bed from pulmonary arteries down to the capillary level and pulmonary veins remained intact. SEM micrographs of decellularized lung showed preserved bronchial, alveolar and vascular basement membranes with no evidence of retained cells. The meshwork of elastic and reticular fibers providing the major structural support to the interalveolar septum as well as the septal basement membrane were intact, including the dense network of capillaries within the pulmonary interstitium.

SEM micrographs of the decellularized trachea showed intact ECM architecture with decellularized hyaline cartilage rings and a rough luminal basal membrane without respiratory epithelium.

### Example 4—Decellularization of Rat Liver

For liver isolation, the caval vein was exposed through a median laparotomy, dissected and cannulated using a mouse aortic cannula (Radnoti Glass. Monrovia, Calif.). The hepatic artery and vein and the bile duct were transected and the liver was carefully removed from the abdomen and submerged in sterile PBS (Hyclone, Logan, Utah) to minimize pulling force on portal vein, 15 minutes of heparinized PBS perfusion was followed by 2-12 hours of perfusion with 1% SDS (Invitrogen, Carlsbad, Calif.) in deionized water and 15 minutes of 1% Triton-X (Sigma, St. Louis, Mo.) in deionized water. The liver was then continuously perfused with antibiotic containing PBS (100 U/ml penicillin-G (Gibco, Carlsbad, Calif.), 100 U/ml streptomycin (Gibco, Carlsbad, Calif.), 0.25 μg/ml Amphotericin B (Sigma, St. Louis, Mo.)) for 124 hours.

120 minutes of SDS perfusion followed by perfusion with Triton-X 100 were sufficient to generate a completely decellularized liver. Movat pentachrome staining of decellularized liver confirmed retention of characteristic hepatic organization with central vein and portal space containing hepatic artery, bile duct and portal vein.

### Example 5—Methods and Materials Used to Evaluate the Decellularized Organs

Histology and Immunofluorescence. Movat Pentachrome staining was performed on paraffin embedded decellularized tissues following the manufacturers instructions (American Mastertech Scientific, Lodi, Calif.). Briefly, deparaffinized slides were stained using Verhoeff's elastic stain, rinsed, differentiated in 2% ferric chloride, rinsed, placed in 5% sodium thiosulfate, rinsed, blocked in 3% glacial acetic acid, stained in 1% alcian blue solution, rinsed, stained in crocein scarlet-acid fuchsin, rinsed, dipped in 1% glacial acetic acid, destained in 5% phosphotungstic acid, dipped in 1% glacial acetic acid, dehydrated, placed in alcoholic saffron solution, dehydrated, mounted and covered.

Immunofluorescence staining was performed on decellularized tissues. Antigen retrieval was performed on paraffin-embedded tissue (recellularized tissue) but not on frozen sections (decellularized tissue) as follows: Paraffin sections were de-waxed and re-hydrated by 2 changes of xylene for 5 minutes each, followed by sequential alcohol gradient and rinsing in cold running tap water. The slides were then placed in antigen retrieval solution (2.94 g tri-sodium citrate, 22 ml of 0.2 M hydrochloric acid solution, 978 ml ultra-pure water, and adjusted to a pH of 6.0) and boiled for 30 minutes. After rinsing under running cold tap water for 10 minutes, immunostaining was begun. Frozen sections were fixed with 4% paraformaldehyde (Electron Microscopy Sciences, Hatfield, Pa.) in 1× PBS (Mediatech, Herndon, Va.) for 15 minutes at room temperature before staining. Slides were blocked with 4% Fetal Bovine Serum (FBS; Hyclone, Logan, Utah) in 1× PBS for 30 minutes at room temperature. Samples were sequentially incubated for one hour at room temperature with diluted primary and secondary antibodies (Ab). Between each step, slides were washed 3 times (5-10 min each) with 1× PBS. Primary Ab against Collagen I (goat polyclonal IgG (Cat. No. sc-8788), Santa Cruz Biotechnology Inc., Santa Cruz, Calif.), Collagen III (goat polyclonal IgG (Cat. No. sc-2405), Santa Cruz Biotechnology Inc., Santa Cruz, Calif.), Fibronectin (goat polyclonal IgG (Cat. No. sc-6953), Santa Cruz Biotechnology Inc., Santa Cruz, Calif.), and Laminin (rabbit polyclonal IgG (Cat. No. sc-20142), Santa Cruz Biotechnology Inc., Santa Cruz, Calif.) were used at a 1:40 dilution with blocking buffer. Secondary Ab's bovine anti-goat IgG phycoerythin (Cat. No. sc-3747, Santa Cruz Biotechnology Inc., Santa Cruz, Calif.) and bovine anti-rabbit IgG phycoerythin (Cat. No. sc-3750, Santa Cruz Biotechnology Inc., Santa Cruz, Calif.) were used at a 1:80 dilution with blocking buffer. Slides were covered with cover glass (Fisherbrand 22×60, Pittsburgh, Pa.) in hardening mounting medium containing 4′,6-diamidino-2-phenylindole (DAPI) (Vectashield, Vector Laboratories, Inc., Burlingame, Calif.). Images were recorded using ImagePro Plus 4.5.1 (Mediacybernetics, Silver Spring, Md.) on a Nikon Eclipse TE200 inverted microscope (Fryer Co. Inc., Huntley, Ill.) using ImagePro Plus 4.5.1 (Mediacybermetics, Silver Spring, Md.).

Scanning Electron Microscopy. Normal and decellularized tissues were perfusion fixed with 2.5% glutaraldehyde (Electron Microscopy Sciences, Hatfield, Pa.) in 0.1 M cacodylate buffer (Electron Microscopy Sciences, Hatfield, Pa.) for 15 minutes. Tissues were then rinsed two times in 0.1 M cacodylate buffer for 15 minutes. Post-fixation was performed with 1% osmium tetroxide (Electron Microscopy Sciences, Hatfield, Pa.) for 60 minutes. Tissue samples were then dehydrated in increasing concentrations of EtOH (50% for 10 minutes, 70% for 10 minutes two times, 80% for 10 minutes, 95% for 10 minutes two times, 100% for 10 minutes two times). Tissue samples then underwent critical point drying in a Tousimis Samdri-780A (Tousimis, Rockville, Md.). Coating was performed with 30 seconds of Gold/Palladium sputter coating in the Denton DV-502A Vacuum Evaporator (Denton Vacuum, Moorestown, N.J.). Scanning electron microscopy images were taken using a Hitachi S4700 Field Emission Scanning Electron Microscope (Hitachi High Technologies America, Pleasanton, Calif.).

Mechanical Testing. Crosses of myocardial tissue were cut from the left ventricle of rats so that the center area was approximately 5 mm×5 mm and the axes of the cross were aligned in the circumferential and longitudinal directions of the heart. The initial thickness of the tissue crosses were measured by a micrometer and found to be 3.59±0.14 mm in the center of the tissue cross. Crosses were also cut from decellularized rat left ventricular tissue in the same orientation and with the same center area size. The initial thickness of the decellularized samples was 238.5±38.9 μm. In addition, the mechanical properties of fibrin gels was tested, another tissue engineering scaffold used in engineering vascular and cardiac tissue. Fibrin gels were cast into cross-shaped molds with a final concentration of 6.6 mg of fibrin/ml. The average thickness of the fibrin gels was 165.2±67.3 μm. All samples were attached to a biaxial mechanical testing machine (Instron Corporation, Norwood, Mass.) via clamps, submerged in PBS, and stretched equibiaxiaily to 40% strain. In order to probe the static passive mechanical properties accurately, the samples were stretched in increments of 4% strain and allowed to relax at each strain value for at least 60 seconds. Forces were converted to engineering stress by normalizing the force values with the cross sectional area in the specific axis direction (5 mm×initial thickness). Engineering stress was calculated as the displacement normalized by the initial length. In order to compare the data between the two axes as well as between sample groups, a tangential modulus was calculated as follows:

[T(ε=40% strain)−T(ε=36% strain)]/4% strain

where T is engineering stress and ε is engineering strain. The values for the tangential modulus were averaged and compared between the two axes (circumferential and longitudinal) as well as between groups.

### Example 6—Assessment of Biocompatibility of Decellularized Organ

To assess biocompatibitity, 100,000 mouse embryonic stem cells (mESC) suspended in 1 cc of standard expansion media (Iscove's Modified Dulbecco's Medium (Gibco, Carlsbad, Calif.), 10% Fetal Bovine Serum (Hyclone, Logan, Utah), 100 U/ml penicillin-G (Gibco, Carlsbad, Calif.), 100 U/ml streptomycin (Gibco, Carlsbad, Calif.), 2 mmol/L L-glutamine (Invitrogen, Carlsbad, Calif.), 0.1 mmol/L 2-mercaptoethanol (Gibco, Carlsbad, Calif.) were seeded onto the ECM sections and on control plates without specific growth factor stimulation or feeder cell support. 4′,6-Diamidino-2-phenylindole (DAPI) was added to the cell culture media at a concentration of 10 μg/ml to label cell nuclei and to allow quantification of cell attachment and expansion. Images were recorded under UV-light and phase contrast at baseline, 24, 48 and 72 hours thereafter using ImagePro Plus 4.5.1 (Mediacybernetics, Silver Spring, Md.) on a Nikon Eclipse TE200 inverted microscope (Flyer Co. Inc., Huntley, Ill.).

The decellularized ECM was compatible with cell viability, attachment and proliferation. Seeded mESCs engrafted on the ECM scaffolds and began to invade the matrix within 72 h of cell seeding.

### Example 7—Evaluation of Decellularized Organs

Aortic valve competence and integrity of the coronary vascular bed of SDS decellularized rat heart was assessed by Langendorff perfusion with 2% Evans blue dye. No left ventricular filling with dye was observed, indicating an intact aortic valve. Macroscopically, filling of the coronary arteries up to the fourth branching point was confirmed without signs of dye leakage. In tissue sections, perfusion of large (150 μm) and small (20 μm) arteries and veins was subsequently confirmed by red fluorescence of Evans blue-stained vascular basal membrane.

To confirm the retention of major cardiac ECM components, immunofluorescent staining of SDS decellularized ECM scaffolds was performed. This confirmed the presence of major cardiac ECM components such as collagens I and III, fibronectin and laminin, but showed no evidence of retained intact nuclei or contractile elements including cardiac myosin heavy chain or sarcomeric alpha actin.

Scanning electron micrographs (SEM) of SDS decellularized cardiac ECM demonstrated that fiber orientation and composition were preserved in aortic wall and aortic valve leaflet with an absence of cells throughout the entire tissue thickness. Decellularized left and right ventricular wall retained ECM fiber composition (weaves, struts, coils) and orientation, while myofibers were completely removed. Within the retained ECM of both ventricles, intact vascular basal membranes of different diameters without endothelial or smooth muscle cells were observed. Furthermore, a thin layer of dense epicardial fibers underneath an intact epicardial basal lamina was retained.

To assess mechanical properties of decellularized heart tissue, bi-axial testing was performed and compared to fibrin gels, which is frequently used as an artificial ECM scaffold in cardiac tissue engineering. The normal rat ventricle and decellularized samples were highly anisotropic with respect to the stress-strain behavior. Conversely, in the fibrin gel sample, the stress-strain properties were extremely similar between the two principal directions. The directional dependence of stress-strain behavior was present in all samples in the normal rat ventricle and decellularized groups, and the isotropic nature of the stress-strain properties was typical of all samples in the fibrin gel group.

In order to compare the stress-strain properties between these two groups and also between the principal axes of the hearts, a tangential modulus was calculated at 40% strain (see Example 5 for the equation) in both the circumferential and longitudinal direction. Note that in both directions, the decellularized sample group had a significantly higher modulus than the normal rat ventricle and fibrin gel sample groups. There was a significant difference, however, between the moduli in the two directions for both the normal rat ventricle and the decellularized matrix, but not for the fibrin gel.

For the intact left ventricular tissue, the stress at 40% strain varied between 5 and 14 kPa in the longitudinal direction and between 15 and 24 kPa in the circumferential direction, which is in agreement with previously published data. In both the rat ventricular tissue and the decellularized rat ventricular tissue, the circumferential direction was stiffer than the longitudinal direction, most likely due to muscle fiber orientation of the heart. While the fiber orientation changes through the thickness of the cardiac tissue, the majority of the fibers were oriented in the circumferential direction and thus, this direction would be expected to be stiffer. The decellularized tissue was significantly stiffer than the intact tissue. This also would be expected since the extracellular matrix is stiffer than the cells themselves, and the combination of ECM and cells would likely not be as stiff as just the ECM alone. While the values of the tangential modulus of the decellularized tissue seem rather large, they are only slightly greater than values of the Young's modulus for purified elastin (approximately 600 kPa) and less than Young's modulus of a single collagen fiber (5 Mpa), placing the values determined herein within a reasonable range.

### Example 8—Decellularization of Other Organs or Tissues

In addition to rat heart, lung, kidney and liver, similar results were generated by applying the perfusion decellularization protocol described herein to skeletal muscle, pancreas, small and large bowel, esophagus, stomach, spleen, brain, spinal cord and bone.

### Example 9—Decellularization of Pig Kidney

Pig kidneys were isolated from heparinized male animals. To allow perfusion of the isolated organs, the renal artery was cannulated and blood was washed out with PBS perfusion over 15 minutes. Perfusion with 27 L of 1% SDS in deionized water was performed for 35.5 hours at a pressure of 50-100 mmHg. Perfusion with 1% Triton-X-100 in deionized water was initiated to remove SDS from the ECM scaffold. Washing and buffering of the decellularized kidneys was then performed by perfusion with antibiotic containing PBS for 120 hours to remove detergents and obtain a biocompatible pH.

Organ clearing was observed within two hours of initiating perfusion. Clear white color predominated 12 hours into perfusion. Decellularization was terminated with the organ was white semi-transparent.

### Example 10—Transplantation of Decellularized Heart

Hearts from F344 rats were prepared by cannulating the aorta distal to the Ao valve and ligating all other great vessels and pulmonary vessels except the left branch of the pulmonary trunk (distal to its bifurcation) and the inferior vena cava (IVC). Decellularization was achieved using Langendorf retrograde coronary perfusion and 2 liters of 1% SDS over 12-16 hours. The hearts were then renatured with 35 mL of 1% Triton-X-100 over 30-40 minutes, and then washed with antibiotic and antifungal-containing PBS for 72 hours. The IVC was ligated before the transplantation.

A large (380 to 400 gram) RNU rat was prepared for reception of the decellularized heart. A blunt-angled mosquito clamp was applied to both the IVC and the abdominal Ao of the host animal to ensure isolation of areas of anastomosis. The aorta of the decellularized heart was anastomosed to the host abdominal aorta proximal and inferior to the renal branches using 8-0 silk suture. The left branch of the decellularized heart's pulmonary trunk was anastomosed to the closest region of the host IVC to minimize physical stress on pulmonary trunk.

After both vessels were sewn into the host animal, the clamp was released and the decellularized heart filled with the host animal's blood. The recipient animal's abdominal aortic pressure was observed visually in the decellularized heart and aorta. The decellularized heart became distended and red with blood. Bleeding was minimal at the site of anastomosis. Heparin was administered 3 minutes after clamp release (initiation of perfusion), and the heart was photographed and positioned in the abdomen to minimize stress on the sites of anastomosis. The abdomen was closed in sterile fashion and the animal monitored for recovery. At 55 hours post-transplant, the animal was euthanized and the decellularized heart was explanted for observation. The animals that did not receive heparin showed a large thrombosis in the LV upon dissection and evaluation. Blood was also observed in coronary arteries in both the right and left sides of the heart.

In other transplant experiments, the clamp was released after both vessels were sewn into the host animal, and the decellularized heart filled with the host animal's blood. The recipient animal's abdominal aortic pressure was observed visually in the decellularized heart and aorta. The decellularized heart became distended and red, and bleeding was minimal at the site of anastomosis. Heparin was administered (3000 IU) by IP injection 3 minutes after clamp release (initiation of perfusion). The heart was photographed and positioned in the abdomen to minimize stress on the sites of anastomosis. The abdomen was closed in sterile fashion and the animal monitored for recovery. The animal was found dead from hemorrhage at approximately 48 hours after transplantation. Transplantation time is currently in the 55 to 70 minute range.

### Section C. Recellularization

### Example 1—Recellularization of Cardiac ECM Slices

To evaluate biocompatibility of decellularised ECM, 1 mm thick slices of one decellularised heart were cultured with myogenic and endothelial cell lines. 2×10⁵ rat skeletal myoblasts, C2C12 mouse myoblasts, human umbilical cord endothelial cells (HUVECs), and bovine pulmonary endothelial cells (BPEC) were seeded onto tissue sections and co-cultured under standard conditions for 7 days. Myogenic cells migrated through and expanded within the ECM and aligned with the original fiber orientation. These myogenic cells showed increased proliferation and fully re-populated large portions of the ECM slice. Endothelial cell lines showed a less invasive growth pattern, forming a monolayer on the graft surface. There were no detectable antiproliferative effects under these conditions.

### Example 2—Recellularisation of Cardiac ECM by Coronary Perfusion

To determine the efficiency of seeding regenerative cells onto and into decellularised cardiac ECM by coronary perfusion, a decellularized heart was transferred to an organ chamber and continuously perfused with oxygenised cell culture media under cell culture conditions (5% CO₂, 60% humidity, 37° C.). 120×10⁶ PKH labelled HUVECs (suspended in 50 ml of endothelial cell growth media) were infused at 40 cm H₂O coronary perfusion pressure. Coronary effluent was saved and cells were counted. The effluent was then recirculated and perfused again to deliver a maximum number of cells. Recirculation was repeated two times. After the third passage, approximately 90×10⁶ cells were retained within the heart. The heart was continuously perfused with 500 ml of recirculating oxygenised endothelial cell culture media for 120 hours. The heart was then removed and embedded for cryosectioning. HUVECs were confined to arterial and venous residues throughout the heart, but were not yet completely dispersed throughout the extravascular ECM.

### Example 3—Recellularization of a Decellularized Rat Heart with Neonatal Rat Heart Cells

Isolation and preparation of rat neonatal cardiocytes. On day one, eight to ten SPF Fisher-344 neonatal pups, aged 1-3 days (Harlan Labs, Indianapolis, Ind.), were sedated with 5% inhaled Isoflurane (Abbott Laboratories, North Chicago, Ill.), sprayed with 70% EtOH, and a rapid sternotomy was performed in sterile fashion. Hearts were excised and placed immediately into 50 ml conical tube on ice containing HBSS; Reagent #1 from a neonatal cardiomyocyte isolation system (Worthington Biochemical Corporation, Lakewood, N.J.). Supernatant was removed and whole hearts were washed once with cold HBSS by vigorous swirling. Hearts were transferred to a 100 mm culture dish containing 5 ml cold HBSS, the connective tissue was removed, and remaining tissue was minced into pieces <1 mm². Additional HBSS was added to bring total plate volume to 9 ml, to which 1 ml Trypsin (Reagent #2, Worthington kit) was added to give a final concentration of 50 μg/ml. Plates were incubated overnight in a 5° C. cooler.

On day two, the plates were removed from the cooler and placed in a sterile hood on ice. Tissue and trypsin-containing buffer were transferred to 50 ml conical tubes on ice using wide-mouth pipettes. Trypsin Inhibitor (Reagent #3) was reconstituted with 1 ml HBSS (Reagent #1) and added to the 50 ml conical tube and gently mixed. The tissue was oxygenated for 60-90 seconds by passing air over the surface of the liquid. The tissue was then warmed to 37° C. and collagenase (300 units/ml) reconstituted with 5 ml Leibovitz L-15 was added slowly. The tissue was placed in a warm (37° C.) shaker bath for 45 minutes. Next, the tissue was titrated ten times using a 10 ml pipet to release the cells (3 mls per second) and then strained through a 0.22 μm filter. The tissue was washed with an 5 additional mls of L-15 media, titrated a second time, and collected in the same 50 ml conical tube. The solution of cells was then incubated at room temperature for 20 minutes, and spun at 50×g for five minutes to pellet the cells. The supernatant was gently removed and the cells were resuspended in the desired volume using Neonatal-Cardiomyocyte Media.

Media and Solutions. All media were sterile filtered and stored in the dark in 5° C. coolers. Worthington Isolation kit contains a suggested media, Leibovitz L-15 for culture. This media was used for Day Two of the tissue processing only. For plating, an alternate calcium-containing media was used, which is described herein. Worthington Leibovitz L-15 Media: Leibovitz media powder was reconstituted using 1 L cell-culture grade water. Leibowitz L-15 media contains 140 mg/m.1 CaCl, 93.68 mg/ml MgCl, and 97.67 mg/ml MgS. Neonatal-Cardiomyocite Media: Iscove's Modified Dulbecco's Medium (Gibco, Cat. No. 12440-053) was supplemented with 10% Fetal Bovine Serum (Hyclone), 100 U/ml penicillin-G (Gibco), 100 U/ml streptomycin (Gibco), 2 mmol/L L-glutamine (Invitrogen), and 0.1 mmol/L 2-mercaptoethanol (Gibco, Cat. No. 21985-023) and sterile filtered before use. Amphotericine-B was added as needed (0.25 μg/ml final concentration). This media was enhanced with 1.2 mM CaCl (Fisher Scientific, Cat. No. C614-500) and 0.8 mM MgCl (Sigma, Cat. No. M-0250).

In Vitro Culture Analysis of Recellularization. As a step towards creating a bioartificial heart, the isolated ECM was recellularized with neonatal heart-derived cells. Completely decellularized hearts (made as described herein) were injected with a combination of 50×10⁶ freshly isolated rat neonatal cardiomyocytes, fibrocytes, endothelial and smooth muscle cells. The heart tissue was then sliced and the slices were cultured in vitro to test the biocompatibility of the decellularized ECM and the ability of the resulting constructs to develop into myocardium rings.

Minimal contractions within the resulting rings were observed microscopically after 24 hours, demonstrating that the transplanted cells were able to attach and engraft on the decellularized. ECM. Microscopically, cells oriented along the ECM fiber direction. Immunofluorescence staining confirmed the survival and engraftment of cardiomyocytes expressing cardiac myosin heavy chain. Within four days, clusters of contracting cell patches were observed on the decellularized matrix, which progressed to synchronously contracting tissue rings by day 8.

At day 10, these rings were mounted between two rods to measure contractile force under different preload conditions. The rings could be electrically paced up to a frequency of 4 Hz and created contractile force of up to 3 mN under a preload of up to 0.65 g. Thus, with this in vitro tissue culture approach of recellularization, contractile tissue was obtained that generated an equally effective force as that generated by optimized engineered heart tissue rings using artificial ECM constructs.

Recellularization of a Decellularized Heart via Perfusion. Recellularized (50×10⁶ freshly isolated rat neonatal cardiomyocytes, fibrocytes, endothelial and smooth muscle cells) scaffolds were mounted in a perfusable bioreactor (n=10) that simulated rat cardiac physiology including pulsatile left ventricular distension with gradually increasing preload and afterload (day 1: preload 4-12 mmHg, afterload 3-7 mmHg), pulsatile coronary flow (day 1: 7 ml/min), and electric stimulation (day 2: 1 Hz) under sterile cardiac tissue culture conditions (5% CO₂, 60% H₂O, 37° C.). Perfused organ culture was maintained for one to four weeks. Pressures, flows and EKG were recorded for 30 seconds every 15 minutes throughout the entire culture period. Videos of the nascent bioartificial hearts were recorded at days four, six and ten after cell seeding.

At day 10 after cell seeding, a more in-depth functional assessment was performed including insertion of a pressure probe into the left ventricle to record left ventricular pressure (LVP) and video recording of wall motion as the stimulation frequency was gradually increased from 0.1 Hz to 10 Hz and performed pharmacological stimulation with phenylephrine (PE). The recellularized heart showed contractile response to single paces with spontaneous contractions following the paced contractions with corresponding increases in LVP. After a single pace, the heart showed three spontaneous contractions and then converted to a fibrillatory state. Similar to the stimulated contractions, spontaneous depolarizations caused a corresponding increase in LVP and a recordable QRS complex possibly indicating the formation of a developing stable conduction pattern.

Once stimulation frequency was increased to 0.4 Hz, an average of two spontaneous contractions occurred after each induced contraction; at a pacing frequency up to 1 Hz, only one spontaneous contraction occurred; and at a pacing frequency of 5 Hz, no spontaneous contractions occurred. Maximum capture rate was 5 Hz, which is consistent with a refractory period of 250 ms for mature myocardium. After perfusion with 100 μM of PE, regular spontaneous de-polarizations occurred at a frequency of 1.7 Hz and were coupled with corresponding increases in LVP.

Histological analysis at day 10 revealed cell dispersion and engraftment throughout the entire thickness of the left ventricular wall (0.5-1.2 mm). Cardiomyocytes aligned with the ventricular fiber direction and formed areas of dense, organized grafts resembling mature myocardium and less dense immature grafts similar to developing myocardium. Immunofluorescence staining for cardiac myosin heavy chain confirmed the cardiomyocyte phenotype. A high capillary density was maintained throughout the newly developed myocardium with an average distance between capillaries of approximately 20 μm, which is similar to that reported for mature rat myocardium. Endothelial cell phenotype was confirmed by immunofluorescent staining for vonWillebrand Factor (vWF). Cell viability was maintained throughout the entire graft thickness, indicating sufficient oxygen and nutrient supply through coronary perfusion.

### Section D. Additional Decellularizations and Recellularizations

### Example 1—Rat Liver Isolation Procedure

Each rat was anesthetized with 75 mg per 1 kg body weight of Ketamine and 10 mg per 1 kg body weight of Xylazine. The rat's abdomen was shaved and sterilized with Betadine. The rat was given a large dose of sodium heparin (100 μL heparin (1,000 UI/mL stock) per 100 g body weight) intravenously into the infragastric vein.

While the heparin was taking effect, the bioreactor flask was assembled. Briefly, tygon tubing was attached to a 250 mL flask (ported on the side of the base), and a reducer tubing adaptor was attached to the tubing (to act as a drain during the wash steps described below). While the heparin was taking effect, a catheter with a rubber stopper was assembled; a 12 cc syringe was filled with PBS and a 3-way stop cock was attached to the syringe. An 18 gauge needle was attached to the syringe and pushed through a No. 8 rubber stopper. To ensure that the liver lies flat in the vessel, it is preferred that the needle be kept even with the bottom of the stopper. A short piece of polyethylene tubing (e.g., PE160) with a melted flange was slipped onto the free end of the tubing after it was alcohol sterilized. A small amount of the PBS was pushed through the catheter to flush the alcohol, and a 10 cm petri dish was filled with enough PBS to cover the isolated liver.

After the heparin had circulated, the abdominal skin was cut and the underlying abdominal muscle exposed. A mid-laparotomy was performed, followed by lateral transverse incisions or a midline incision along the abdominal wall followed by retraction to expose the liver. Gently (the Glisson capsule is fragile), the ligaments that attach the liver to the duodenum, stomach, diaphragm and anterior abdominal wall were cut away. The common bile duct, hepatic artery, and portal vein were cut, leaving sufficient length to insert a catheter, and, finally, the supra-hepatic inferior vena cava was cut. The liver was removed by holding onto the remaining attached supra-hepatic inferior vena cava and placing the liver into the petri dish containing PBS. Any remaining ligaments were cut away.

### Example 2—Decellularization of Liver

The prepared catheter was inserted into the portal vein and tied off with proline sutures. The integrity of the line was validated and latent blood was removed from the liver by perfusion using the PBS (without Mg⁺² and Ca⁺²) in the syringe. The liver-rubber stopper was placed into the bioreactor. The flask was placed over a collection reservoir, and a container of 1% SDS (1.6 L) attached via a line of sufficient length to produce a column that generates a maximum pressure of approximately 20 mm Hg. After 2 to 4 hrs of perfusion, the container of 1% SDS was emptied and refilled with an additional 1.6 L of 1% SDS. A total of four batches of 1.6 L of 1% SDS were typically used to perfuse the liver. After decellularization, the liver was clear white in appearance and vascular conduits were visible.

On day two, the SDS reservoir was disconnected and replaced with a 60 ml syringe filled with dH₂O. The water rinse was followed with 60 mL of 1% Triton X-100, which was followed by another 60 mL wash with dH₂O. The rinsed liver was setup for washing, and perfusion was started with PBS with antimicrobials (e.g., penicillin—streptomycin (e.g., Pen-Strep®)) using a small pump (Masterflex at 50% max capacity, which is about 1.5 mL/min). A length of tygon tubing was run from the bioreactor/flask drain to the PBS reservoir. A length of tubing was run through the pump to a 0.8 micron filter attached to the 3 way stopcock on the flask. An 18 gauge needle was attached to the tubing in the PBS reservoir to keep it lower than the input line. After 6 hours, the wash was replaced with fresh PBS w/Pen-Strep at 1× concentration, the 0.8 micron filter was changed, and the organ was washed overnight.

On day three, the washes continued through 2 more changes of 500 ml of PBS w/Pen-Strep at 1× concentration. At each PBS change, the 0.8 micron filter was changed. The third wash was started in the morning and changed after 6 hours, and the final wash was again allowed to proceed overnight. On day four, the liver was ready for recellularization.

Livers washed twice with 1.6 L of 1% SDS had, on average, 14.27% of the DNA remaining, while livers washed four times with 1.6 L of 1% SDS had, on average, 5.36% of the DNA remaining. That is, two washes with 1% SDS removed approximately 86% of the DNA (compared to cadaveric), while four washes with 1% SDS removed approximately 95% of the DNA (compared to cadaveric).

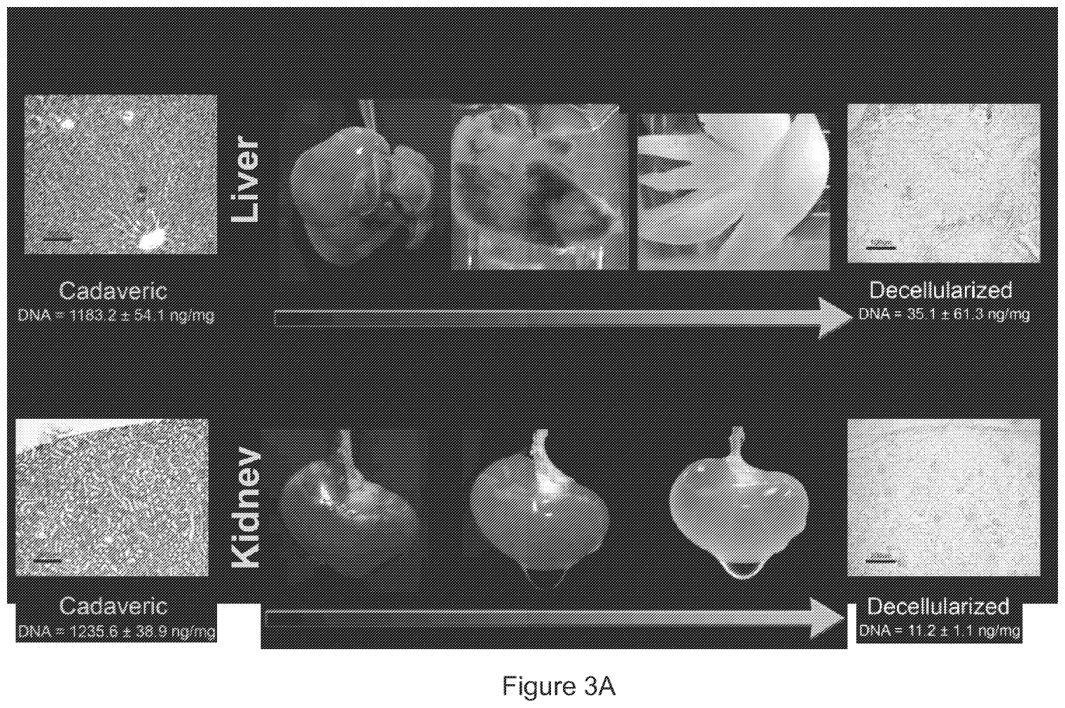

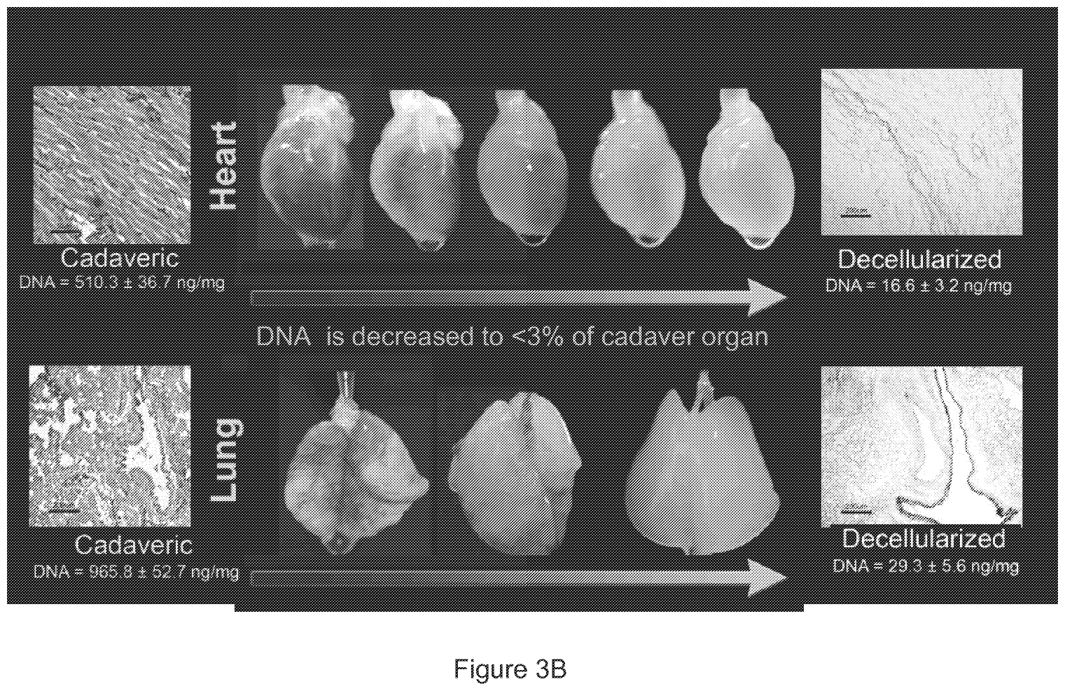

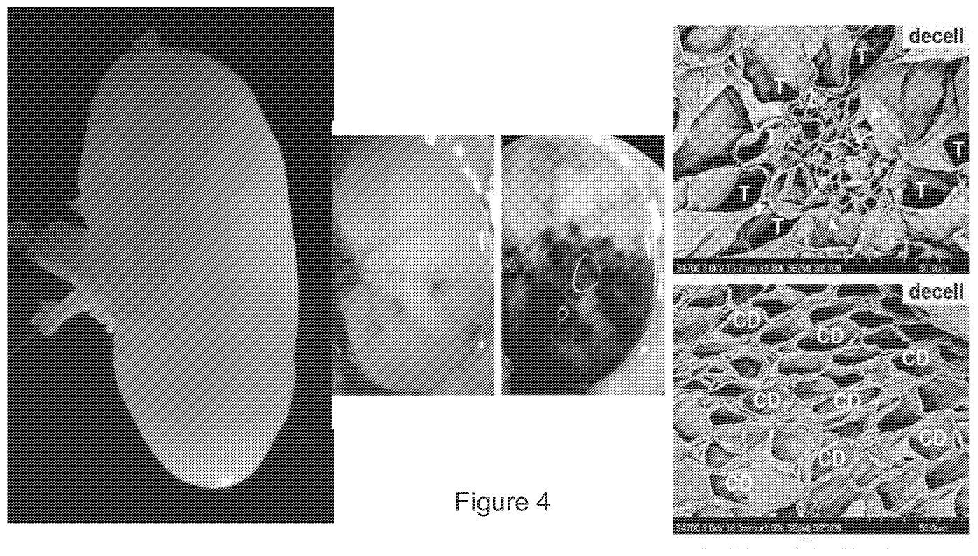

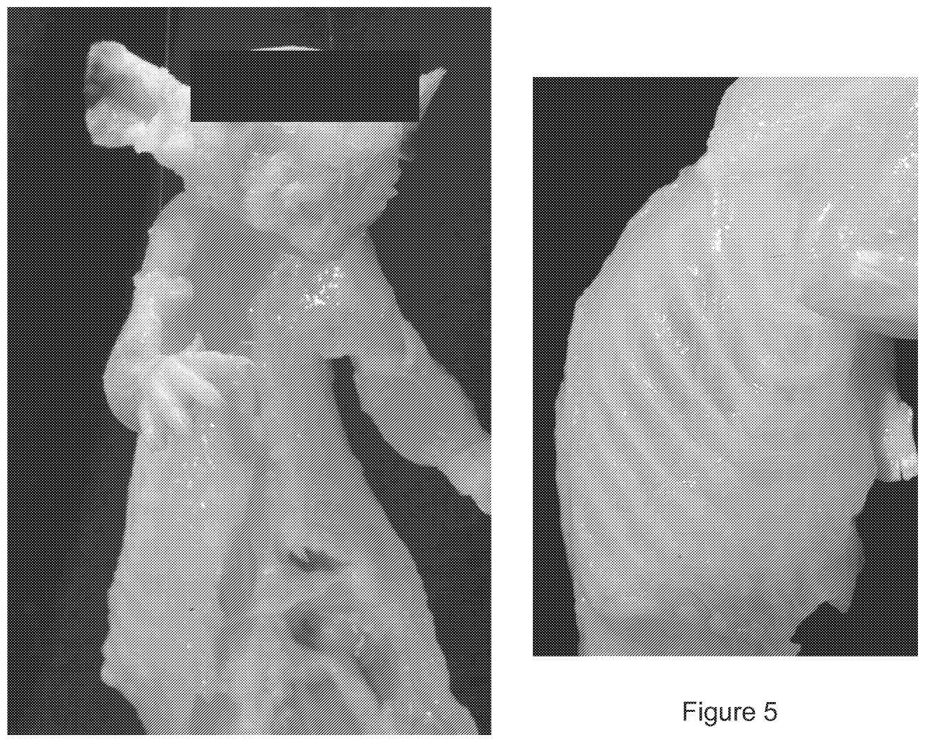

FIG. 3A shows the decellularization of a rat liver as well as a rat kidney and FIG. 3B shows the decellularization of a rat heart and rat lung. The middle portion contains photographs of the progressive decellularization, and the photographs on the right and left are SEM images of the decellularized organ. FIG. 4 shows a decellularized pig kidney and a rat kidney perfused with dye, and also shows EM photos of the glomerulus and the tubules of the decellularized kidney. FIG. 5 shows an entire rat carcass that has been decellularized as described herein.

### Example 3—Recellularization of Liver

Recellularization was performed by suspending cells (40 million primary liver-derived cells or HepG2 human cells) in warmed media (37° C.) at ˜ 8 million cells per milliliter (typically in 5 mL) and loading them into a syringe. The cells were infused via the portal vein while the liver was in the bioreactor or in a petri dish. It is noted that cells also or alternatively can be infused via any other vascular access or directly injected into the parenchyma.

The primary liver-derived cells were obtained by enzymatic digestion of adult rat liver using a Worthington enzyme dissociation kit. Briefly, rat liver was perfused with 1× calcium- and magnesium-free Hanks Balanced Salt Solution (Kit Vial #1) for 10 min at 20 ml/min via portal vein prior to removal from the rat. Next, liver was recirculated with 100 mL of L-15 with MOPS buffer containing enzymes from Kit Vials #2 and #3 (Collagenase (22,500 Units) Elastase (30 Units) and DNase I (1,000 Units)) for 10-15 min at 20 mL/min. This was followed by mechanical disruption of the organ to release cells. Cells were centrifuged at 100 g and re-suspended in culture media twice prior to using for recellularization.

The rate of perfusion was controlled based on visual cues observed during the process (e.g., tension of the perfused liver lobe, escape of cells from the liver, and distribution of cells through the target liver lobe). After recellularization, the liver inside the bioreactor was placed into an incubator at 37° C. and 5% CO₂. An oxygenated media reservoir was attached (containing 50 mL of media); humidified carbogen (95% oxygen, 5% carbon dioxide) was bubbled through the media in the reservoir. A peristaltic pump was used to re-circulate the media (at 37° C.) through the liver at rates ranging from 2-10 mL/min. Recellularized rat livers were maintained with daily media changes for 7 days (although the experiments were simply terminated at that time for convenience). Media was samples and stored at −20° C. during the daily changes to measure albumin and urea. On day 7, cytochrome P-450 assays were performed.

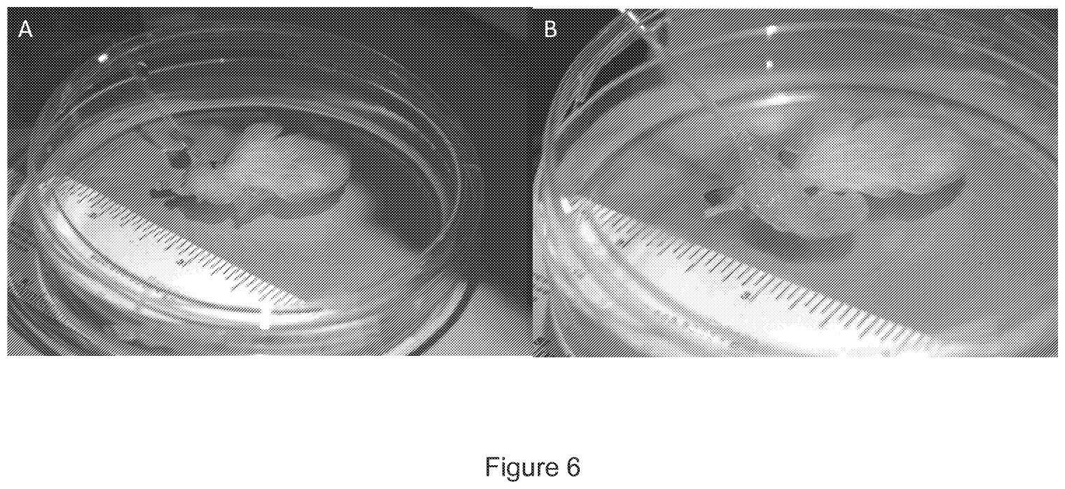

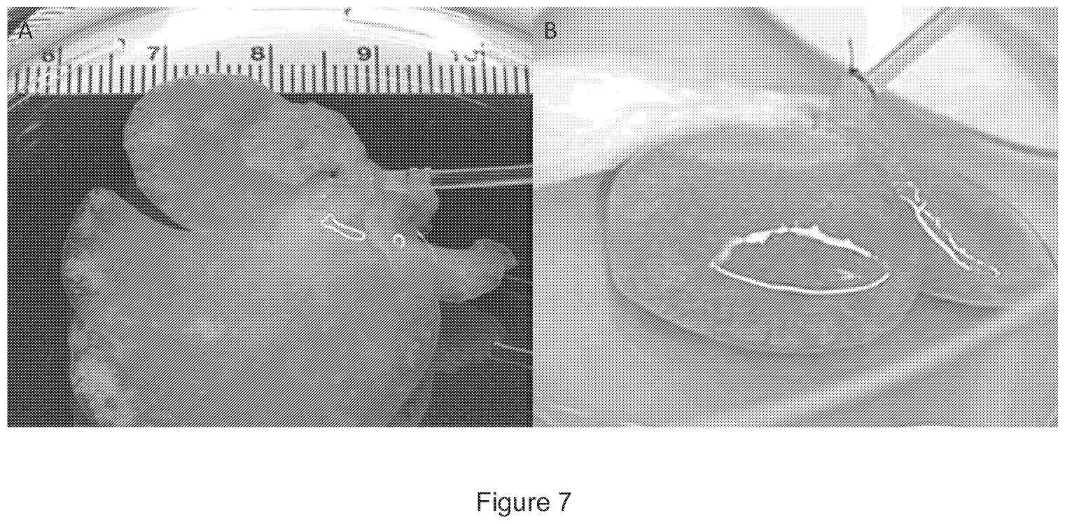

FIG. 6 shows recellularization of a decellularized rat liver. Primary hepatocytes were injected with a syringe into a single lobe via a portal vein catheter. FIG. 7 shows the targeted delivery of primary rat hepatocytes into the caudate lobes (A) or the inferior/superior right lateral lobes (B) of a decellularized rat liver.

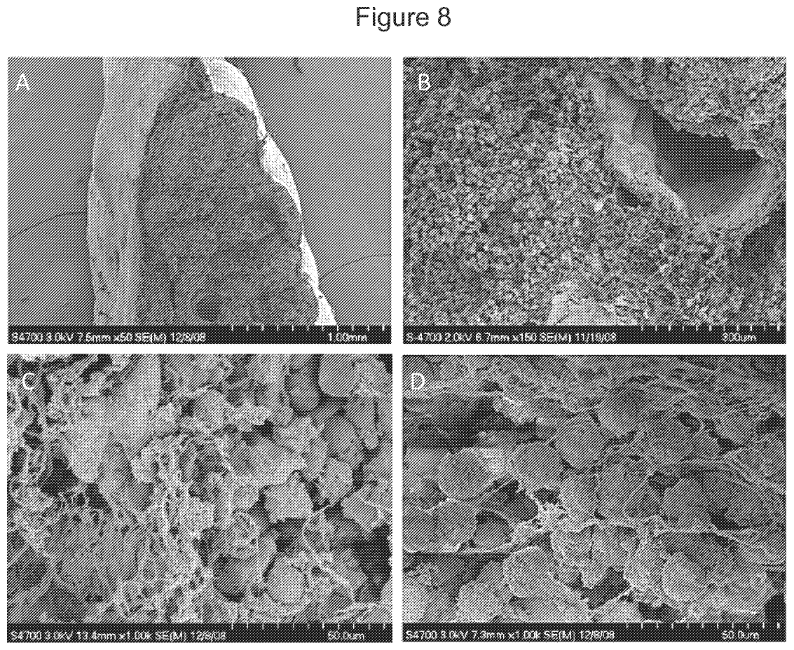

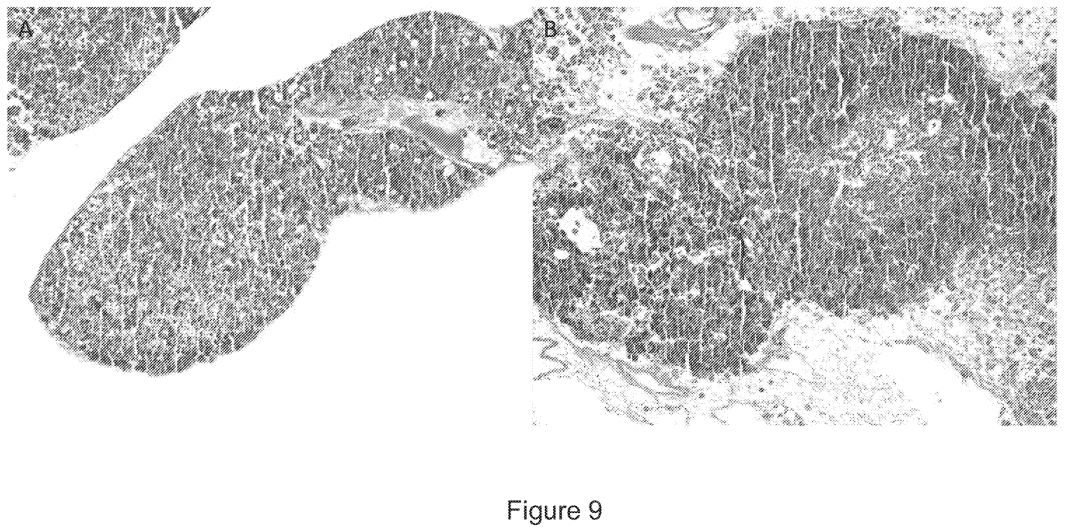

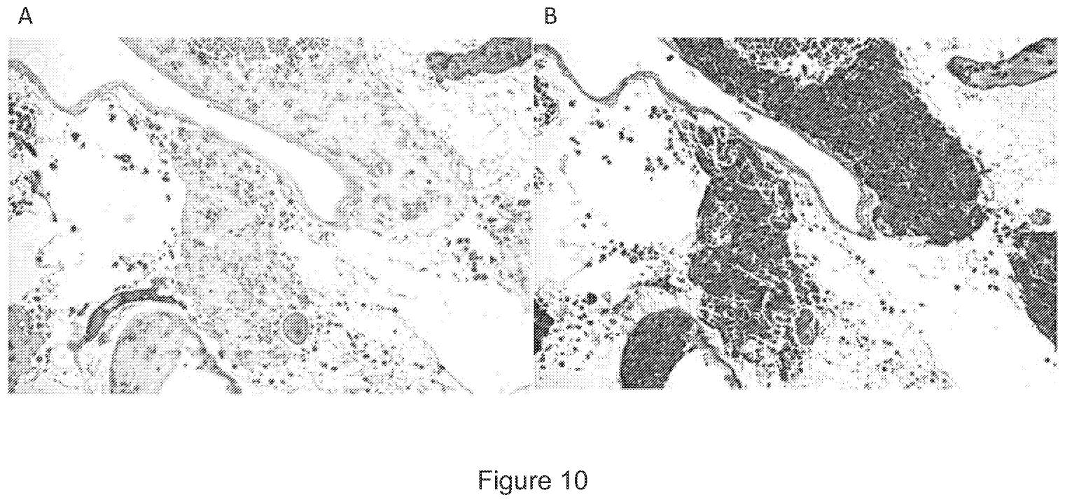

FIG. 8 shows scanning electron micrography (SEM) of recellularized rat liver cultured for 1 week. These data show the similarity of cadaveric liver to recellularized liver at the ultrastructural level. Cells were integrated into the matrix bed and had a similar shape as those in freshly isolated cadaveric tissue. FIG. 9 shows Masson's Trichrome (A) and H&E (B) staining, FIG. 10A shows TUNEL analysis, and FIG. 10B shows Masson's Trichrome staining of recellularized rat liver 1 week following injection of rat hepatocytes into the caudate process. These results demonstrate that the hepatocytes can be delivered to and are retained within the matrix, and can be kept viable with perfusion of nutrients.

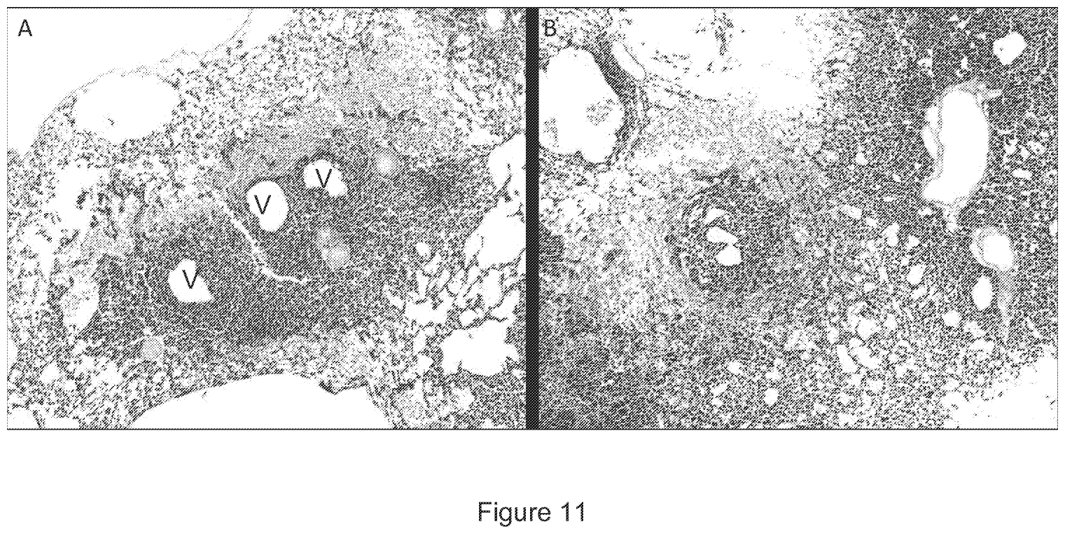

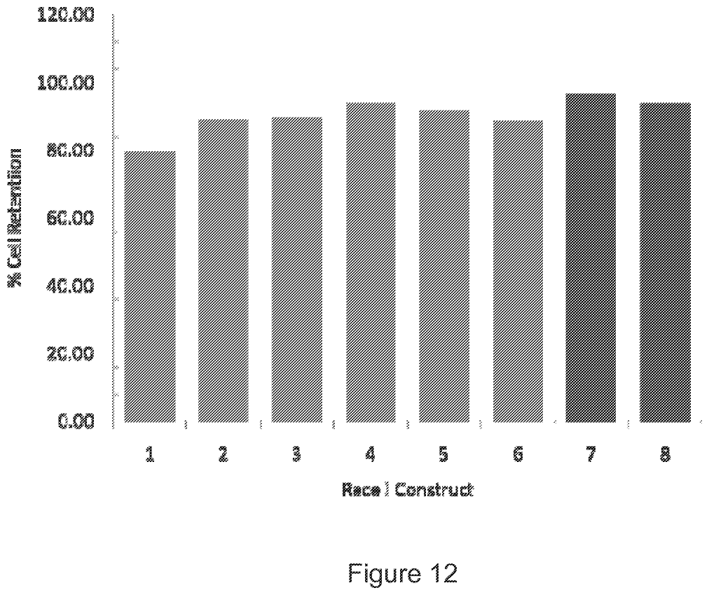

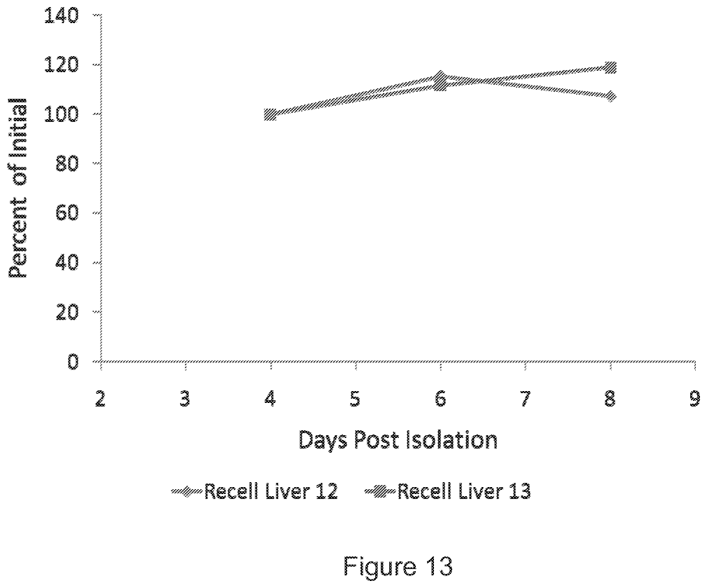

FIG. 11 shows Masson's Trichrome staining of recellularized rat liver one week after injection of human hepatic cell line (HepG2) into the caudate process (A) or the superior/inferior right lateral lobe (B). FIG. 12 is a graph showing the cell retention of primary rat hepatocytes (1-6) and the human HepG2 cell line (7 and 8). Cells were counted before injection for the total number of cells perfused into the liver and the non-adherent cells that flowed through the matrix and ended up in the petri dish were counted; the difference represents the cells retained in the matrix. FIG. 13 is a graph showing that human HepG2 cells remain viable and proliferate after injection into a decellularized rat liver.

### Example 4—Liver Function

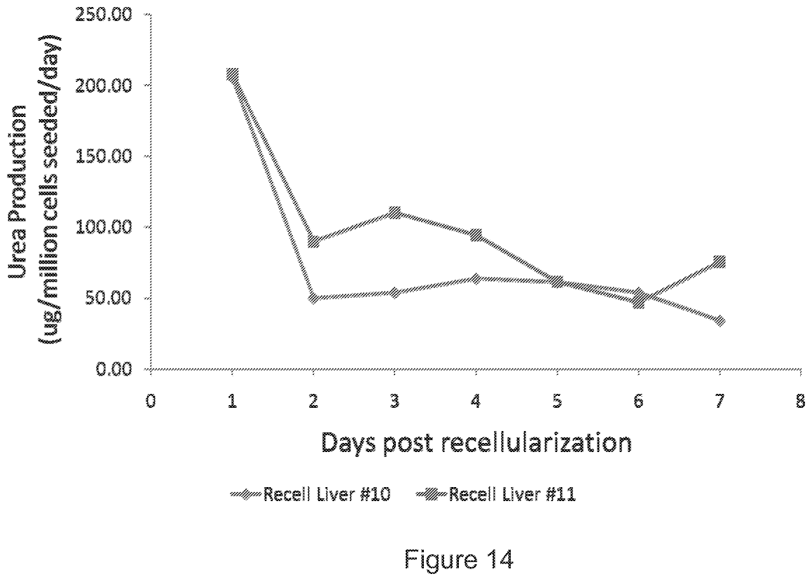

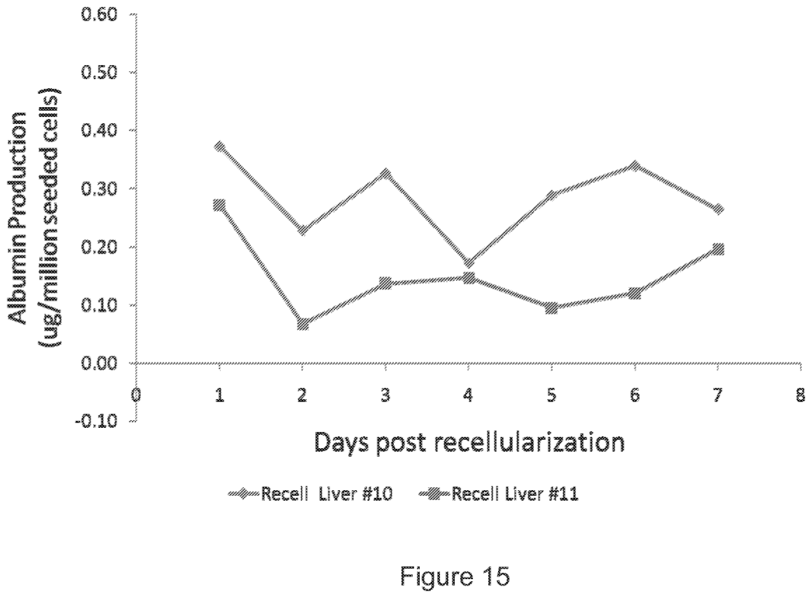

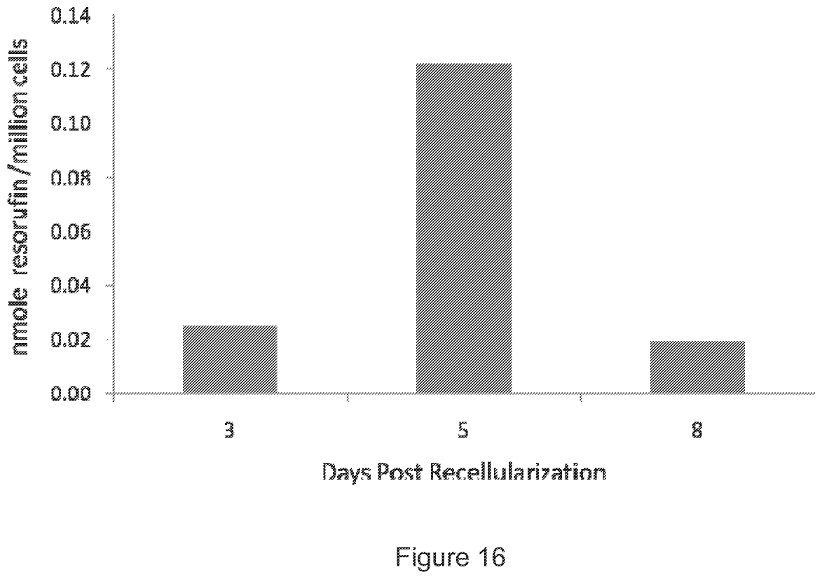

The function of the decellularized and recellularized liver was evaluated as follows. Urea production (FIG. 14), albumin production (FIG. 15), and cytochrome. P-450 IAI (ethoxyresorufin-O-deethylase (EROD)) activity (FIG. 16) were evaluated in a liver recellularized with primary rat hepatocytes. Urea production was determined using a Berthelot/Colormetric assay kit (Pointe Scientific. Inc.), while albumin production and EROD activity were assayed for using methods adapted from Culture of Cells for Tissue Engineering (Vunjak-Novakovic & Freshney, eds., 2006, Wiley-Liss). These experiments demonstrated that the hepatic derived cells retain liver-specific functionality during the culture period.

### Example 5—Cell Viability Following Recellularization

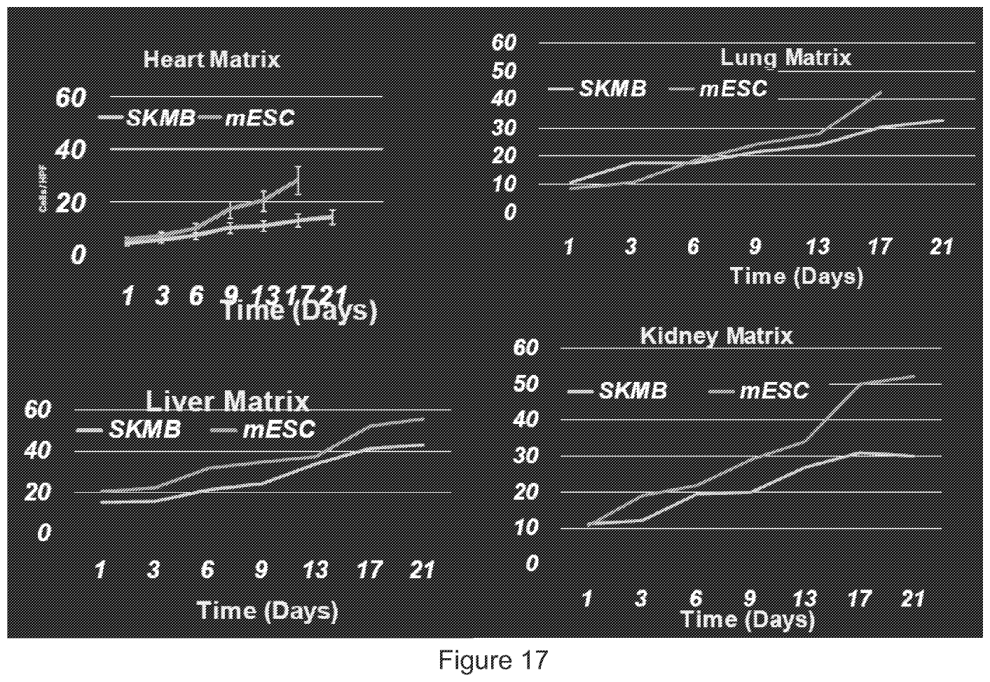

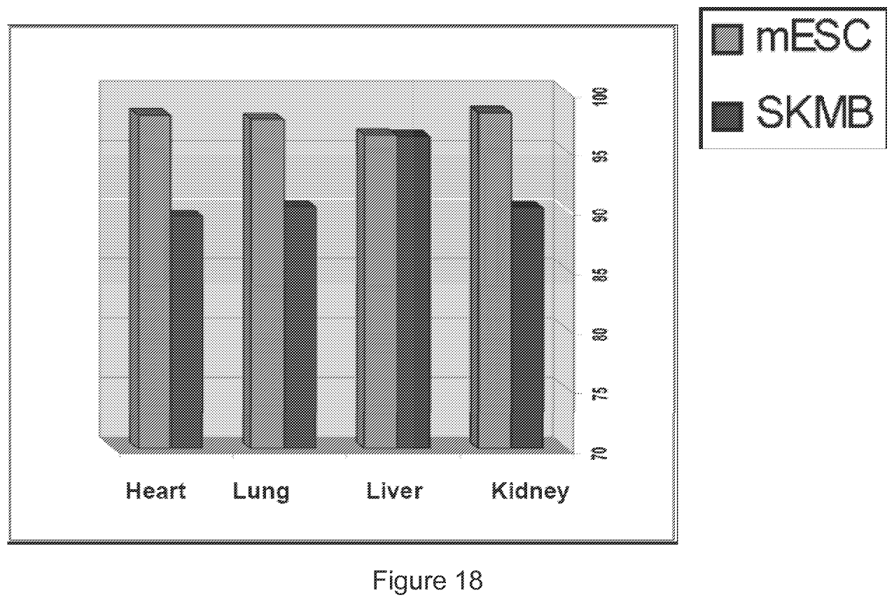

FIG. 17 are graphs showing that embryonic and adult-derived stem/progenitor cells proliferated for at least 3 weeks on decellularized heart, lung, liver, and kidney. Proliferation of cells was determined by counting the number of nuclei DAPI-stained per high power field. FIG. 18 is a graph showing that mouse embryonic stem cells (mESC) and proliferating adult muscle progenitor cells (skeletal myoblasts; SKMB) were viable on decellularized heart, lung, liver, and kidney. Viability of cells was determined using a tunnel assay to detect the degree of apoptosis vs. the number of total DAPI stained cell nuclei after 3 weeks.

Human embryonic stem (ES) cells and human induced pluripotent stem (iPS) cells proliferated for at least 1 week on decellularized heart matrix. Briefly, human ES cells (H9 from WiCell Research Institute; WA09 from National Stem Cell Bank (NSCB)) and an IMR90 subclone of human iPS cells (generated using OCT4, SOX2, NANOG, and LIN28 lentiviral transgenes as described in Zhang et al., 2009, Circ. Res., 104:e30-e41 and obtained from Dr. Timothy Kamp at the University of Wisconsin) were compared on decellularized matrix. H9 cells and iPS cells that contained 20-50% cardiocytes amidst proliferating fibroblasts and other non-beating cells were plated at densities of 200,000 cells and 90,000 cells, respectively, into wells that contained chamber-specific (right or left atria or ventricle) pieces of rat decellularized heart matrix that had been isolated to expose the interior of the matrix. Cells were simply deposited onto the decellularized matrix. Cells were grown in media containing 20% serum for 3 days, and then the serum was reduced to 2% for the next 4 days, consistent with “shifting” proliferating muscle cells toward a beating myocyte phenotype in vitro. Control cells were plated into identical wells coated with gelatin (0.1%) and grown under identical conditions. Cells were grown in EB20 media. Cultures were evaluated by microscopy daily and beating cells were recorded with a video camera. After culturing for a week, a live/dead assay was performed to examine cell viability. In addition, immunohistochemistry is performed to demonstrate the presence of cardiac-related proteins. Cells grown on decellularized matrix were observed to beat by day 3 to 4, whereas cells on gelatin did not beat. By day 5, cells on matrix had expanded and larger areas of beating cells were observed. Beating was sparse or non-existent on cells grown under identical conditions on gelatin.

### Example 6—Recellularization Process

Isolation of Cells

The LV and RV from rat pups were isolated using the Worthington protocol, cutting approximately in the middle of heart. The area from the base to the second LAD branch was discarded, and the remainder placed in ˜10 mL of HBSS. Optionally, the LV and RV portions of the heart can be incubated overnight in Trypsin for up to 18-22 hours at 5° C. After drawing the cells into a syringe for injection into a decellularized matrix, the remaining cells were used as a control (e.g., 10 mL media were added and the cells plated).

### Extracellular Matrix

A well-washed decellularized extracellular matrix (ECM) was obtained. For example, a heart extracellular matrix was washed for 3-4 days using a minimum of 2000 mL PBS solution. The heart was cannulated using 18 ga. Cannulae (IN: LV past mitral valve; OUT: Ao) and secured using 4-0 suture. The LV cannula was advanced near to the apex within the LV lumen (e.g., the tip of the LV cannula was ˜0.7 cm from the Mitral valve). The configuration was checked for the absence of leaks. Optionally, a “High-Speed” test can be performed to ensure secure ECM connections before any cells are introduced by starting the pump into [pre-heart] flow probe range of 25-28 ml/min for at least 5-10 seconds.

### Cell Injections

100-120 mL media was placed into a bioreactor, and a 60 mm culture plate was placed under the apex of the heart to catch excess cells, avoid coronary occlusion, and avoid apoptotic signaling from rogue cells. Cells were injected using 27 ga. needles and 1 cc TB syringes. Approximately 70 μL of cells were injected into the ventricular walls per injection, with a needle entry angle of 15 degrees from normal. Cells were injected into the anterior LV wall 10 to 12 times and into the apex of the heart 3 to 4 times. The total volume of cells injected should be about 1.3 to 1.5 mL. Some backflow and loss of cells is expected. The heart was lowered into the bioreactor, the pump and tank (95% O₂ and 5% CO₂) was turned on, and the heart was monitored for leaks, flow problems, and any other technical problems. The next day, the reactor was opened and pacing leads were attached. Pacing (continuous) was started at Freq: 1 Hz; Delay: 170 MS; Duration: 6 MS; Voltage range: 45-60V; Flow (IN): 18 to 22 mL/min; Flow (OUT): 14-18 mL/min; diff 6 to 7 mL/min.

### Media

The following recipe is for 1 Liter. To IMDM, add 100 mL FBS 10%; 5 mL Pen Strep; 10 mL, L-Glut; 168 μL Amp-B; 1 ml B-Mercap; 20 mL Horse Serum; 180 mg Ca²⁺; 96 mg Mg²⁺; and 50 mg Vitamin C.

### NNCM (NEO) Cells

Neonatal cardiomyocytes (NNCM or NEO cells) were obtained from Worthington kit preps. The NEO cells were temperature sensitive; if they dropped below ˜35° C., they didn't beat as well. The NEO cells started beating on a 2 D plate within 24 hours if not too confluent. As the NEO cells grow and beat together, they grow on top of each other and start to beat in synchrony; eventually, the cells will limit themselves mechanically and stop beating, usually between day 10 and 16.

### Example 7—Structural Comparison of Decellularized and Recellularized Organs with Cadaveric Organs

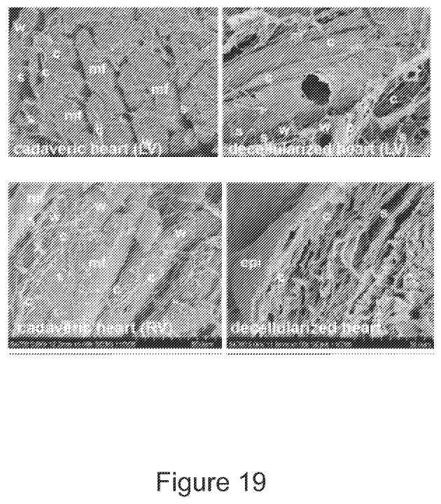

FIG. 19 are SEM photographs of a decellularized heart (right panels) and a cadaveric heart (left panels). SEM photographs were obtained of both the left ventricle (LV) and right ventricle (RV). As can be seen from the photographs, the perfusion-decellularized heart is lacking cellular components but retains spatial and architectural features of the intact myocardium including vascular conduits. In addition, in the perfusion-decellularized matrix, it is possible to see retention of the architectural features including weaves (w), coils (c) and struts (s) within the matrix despite the complete loss of cells.

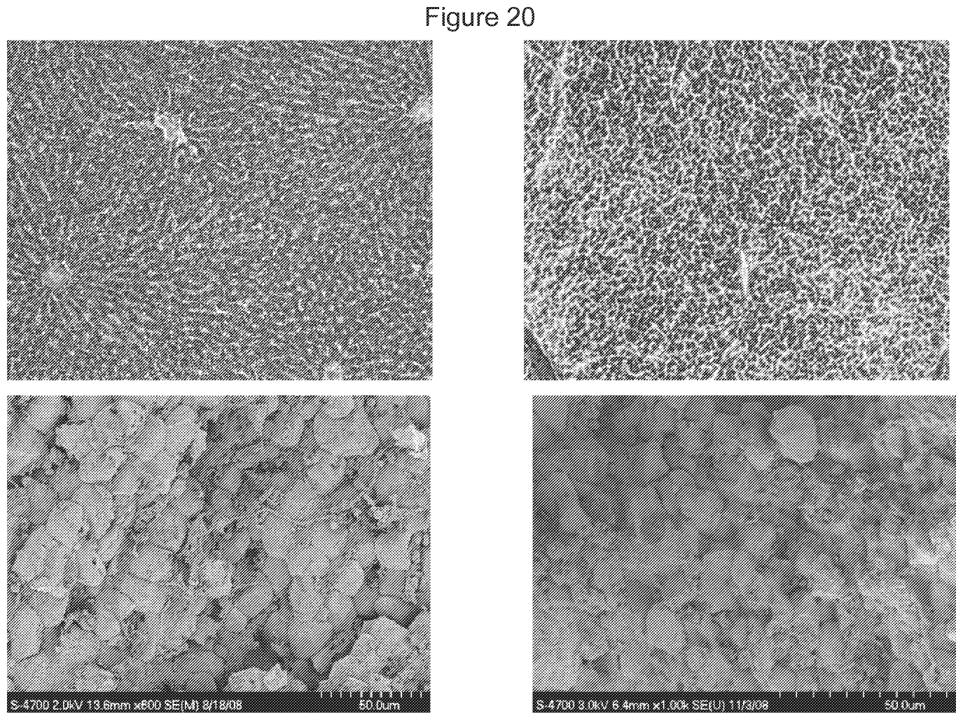

FIG. 20 shows histologic (top) and SEM (bottom) comparison of a rat liver decellularized and recellularized as described herein (right panels) compared to a cadaveric rat liver (left panels). These results illustrate the morphologic similarities and architectural organization between healthy hepatocytes from an intact liver and hepatocytes cultured or seeded on the decellularized liver. The H&E image shows cells in the recellularized liver have begun to organize in a radial fashion around vascular conduits, similar to the architecture seen in freshly isolated healthy (cadaveric) liver. It also illustrates that cells distribute and/or migrate throughout the parenchyma, begin to organize, and are maintained in the matrix for as long as the experiments are continued. The SEM images demonstrate the similarity in cellular organization in the cadaveric and recellularized matrix even at the ultrastructural level.

### Section E. Decellularization by Perfusion vs. Immersion

### Example 1—Decellularization Using Immersion

Organs (rat liver, kidney, heart, lung, muscle, skin, bone, brain and vasculature; porcine liver, gallbladder, kidney and heart) were decellularized using the perfusion methods described herein.

Organs (rat liver, heart and kidney) were decellularized using the immersion methods described in U.S. Pat. Nos. 6,753,181 and 6,376,244. Briefly, an organ was placed in dH₂O and agitated with a magnetic stir bar rotating at 100 rpm for 48 hours at 4° C., and then the organ was transferred to an ammonium hydroxide (0.05%) and Triton X-100 (0.5%) solution for 48 hours with continued magnetic stir bar (100 rpm) stirring of the solution. The solution was changed and the 48 hr immersion with the ammonium hydroxide and Triton X-100 was repeated as needed to decellularize the organ (generally a visual acellular organ). The liver took approximately 5 repetitions of ammonium hydroxide and Triton X-100 to generate a visually acellular organ. After the decellularization process, organs were transferred to dH₂O for 48 hours with agitation (again stirring at 100 rpm); lastly, a final wash was performed with PBS at 4° C. and stirring.

### Example 2—Comparison of Perfusion vs. Immersion

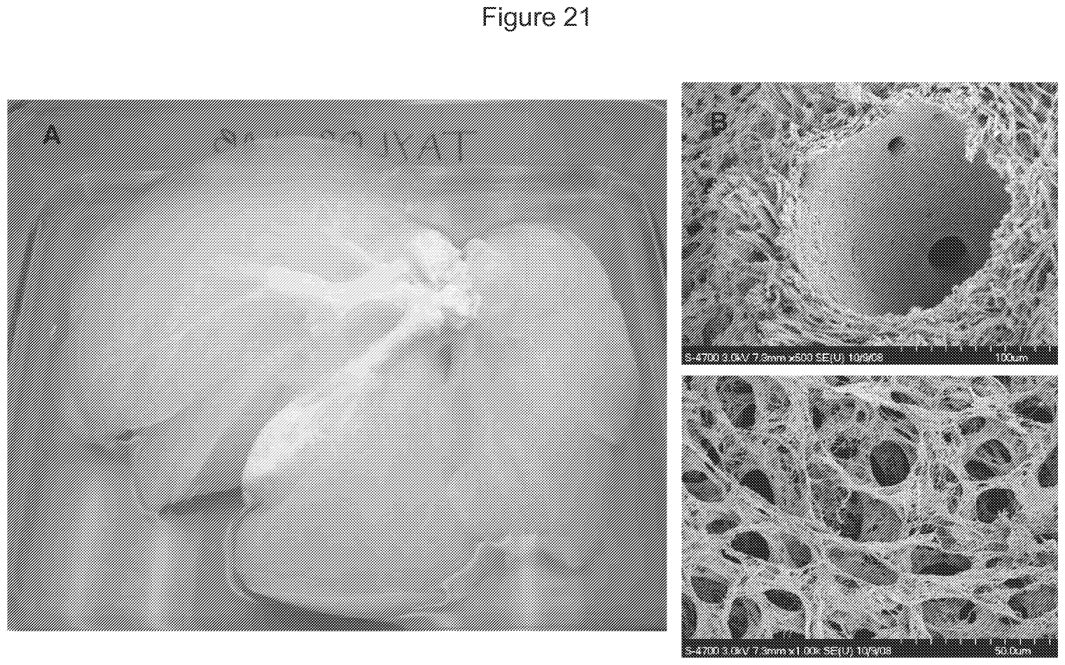

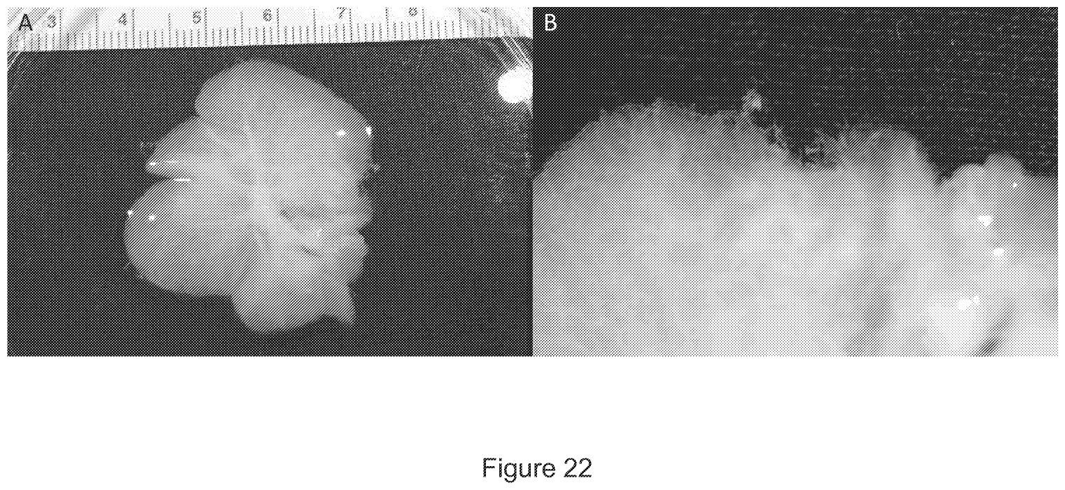

FIG. 21A shows a photograph of a porcine liver that was perfusion decellularized, and FIGS. 21B and 21C show SEM of a vessel and the parenchymal matrix, respectively, of the perfusion decellularized porcine liver. These photographs show the vascular conduits and the matrix integrity of a perfusion decellularized organ. On the other hand, FIG. 22 shows a gross view of an immersion decellularized rat liver, in which fraying of the matrix can be seen at both low (left) and high (right) magnification.

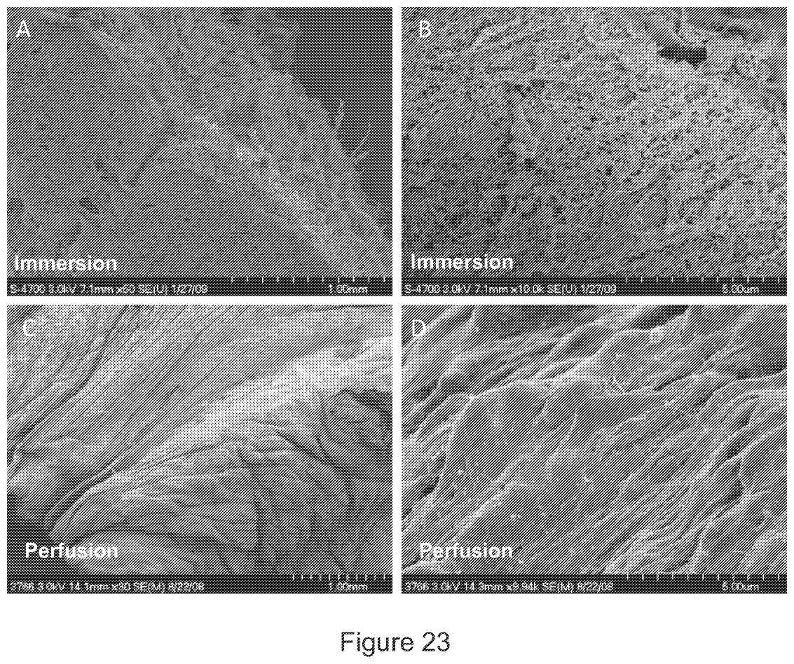

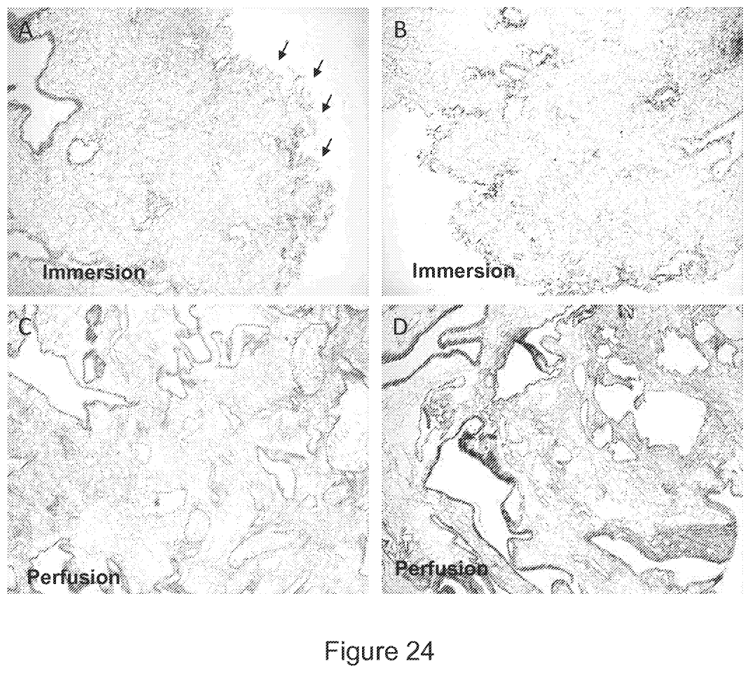

FIG. 23 shows SEM of immersion decellularized rat liver (A and B) and perfusion decellularized rat liver (C and D). These results clearly indicate that immersion decellularization significantly compromised the organ capsule (Glisson's capsule), while perfusion decellularization retained the capsule. In addition, FIG. 24 shows histology of immersion decellularized liver (A, H&E staining; B, Trichrome staining) and perfusion decellularized liver (C, H&E staining; D, Trichrome staining). The immersion decellularized rat liver did not retain cells or dye upon injection.

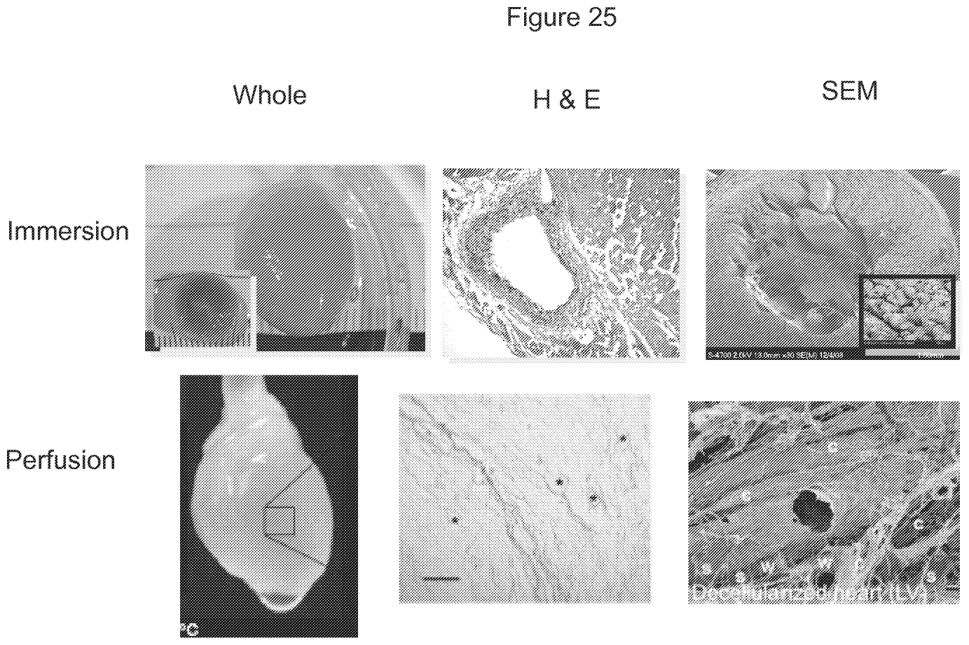

FIG. 25 shows a comparison between immersion decellularization (top row) and perfusion decellularization (bottom row) of a rat heart. The photographs in the left column show the whole organ. As can be seen from the two photographs, the perfusion decellularized organ (bottom left) is much more translucent than the immersion decellularized organ (top left), which retains the iron-rich “brown-red” color of cadaveric muscle tissue and appears to still contain cells. The photographs in the middle column show the H&.E staining pattern of the decellularized tissues. The staining shows that a number of cells, both within the parenchyma and in the walls of the vasculature, remain following immersion decellularization (top middle), while virtually every cell and also the cellular debris is removed following perfusion decellularization (bottom middle) even as patent vascular conduits are evident. In addition, the scanning electron micrographs in the right column show that there is a significant difference in the ultrastructure of the matrix following immersion (top right) vs. perfusion (bottom right) decellularization. Again, complete retention of cellular components throughout the cross section of the myocardium was observed in all the walls of the immersion-decellularized heart, but almost a complete loss of these cellular components was observed in the perfusion-decellularized heart along with the retention of spatial and architectural features of the intact myocardium including vascular conduits. For example, the perfusion-decellularized matrix retained the architectural features within the matrix including weaves (w), coils (c) and struts (s) despite the complete loss of cells.

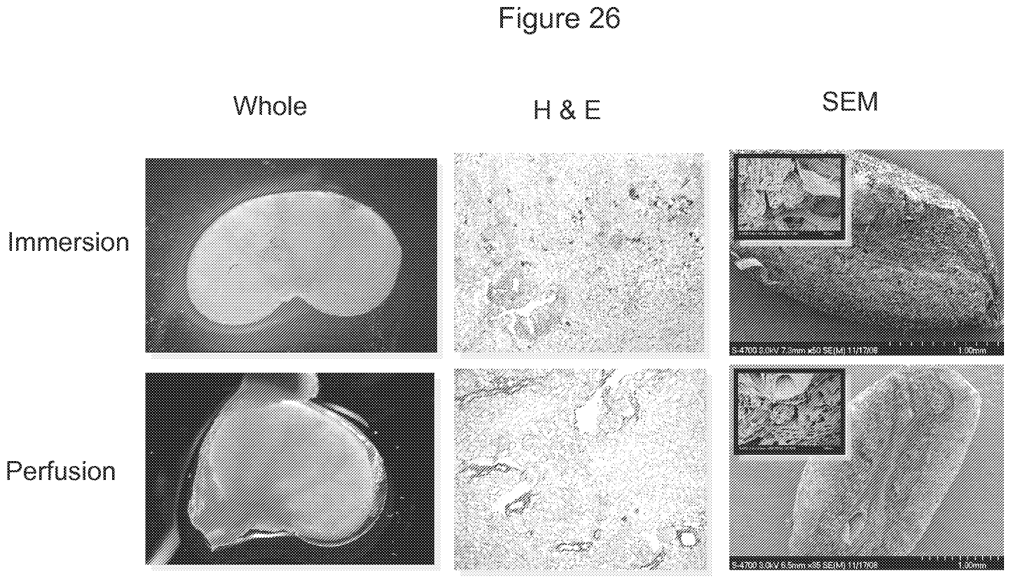

FIG. 26 shows the same comparisons (immersion decellularization (top row) vs. perfusion decellularization (bottom row)) using rat kidney. Unlike heart, the immersion-decellularized whole kidney (top left) looks grossly similar to the perfusion-decellularized whole kidney (bottom left) in that both are fairly translucent. However, in the perfusion-decellularized kidney, the network of vascular conduits within the perfusion-decellularized organ is more obvious and a greater degree of branching can be visualized than in the immersion-decellularised construct. Furthermore, the perfusion-decellularized kidney retains an intact organ capsule, is surrounded by mesentery, and, as shown, can be decellularized along with the attached adrenal gland. The photographs in the center column show the H&E staining pattern of the two tissues. The staining shows that cellular components and/or debris and possibly even intact nuclei (purple stain) remain following immersion-decellularization (top center), while virtually every cell and/or all cellular debris is removed following perfusion-decellularization (bottom center). Likewise, the SEM photographs demonstrate that the immersion-decellularized kidney matrix (top right) suffered much more damage than did the perfusion-decellularized kidney matrix (bottom right). In the immersion-decellularized kidney, the organ capsule is missing or damaged such that surface “holes” or fraying of the matrix are obvious, whereas, in the perfusion decellularized organ, the capsule is intact.

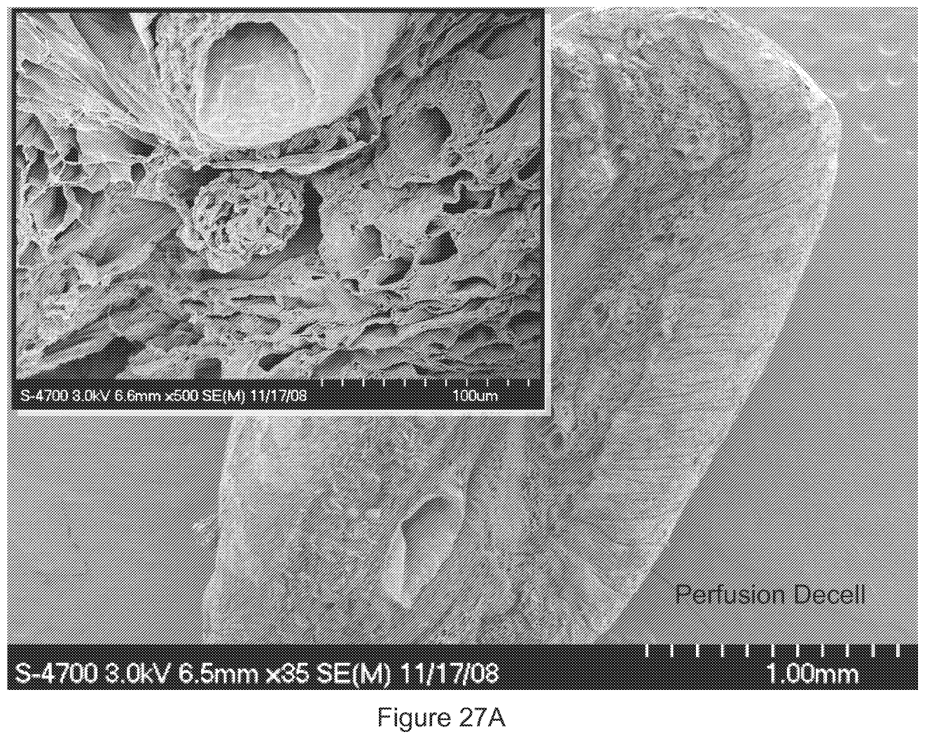

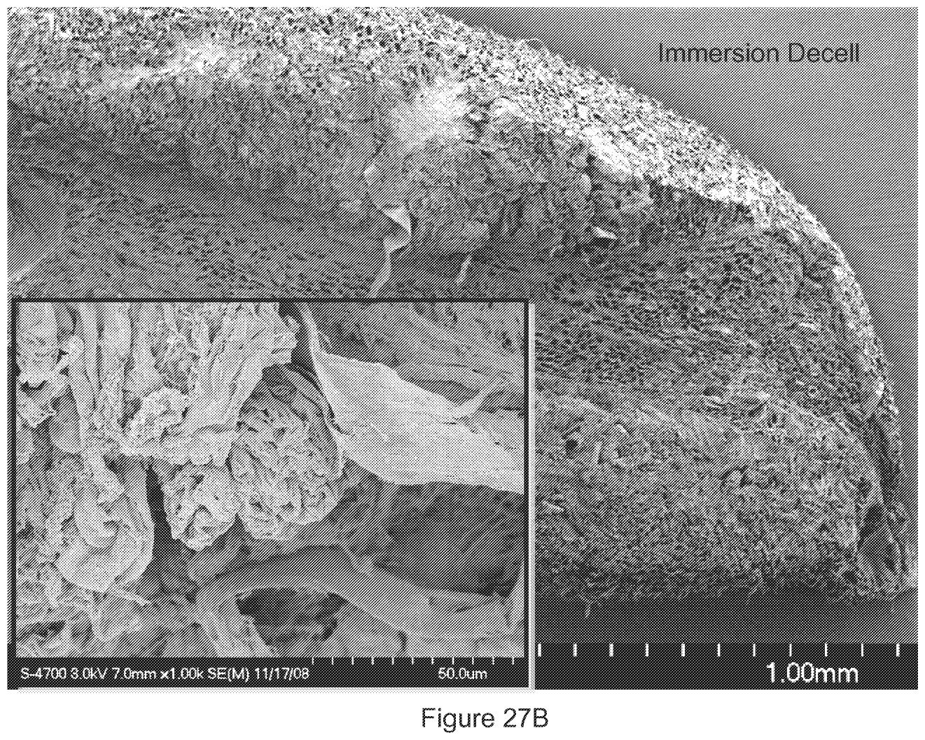

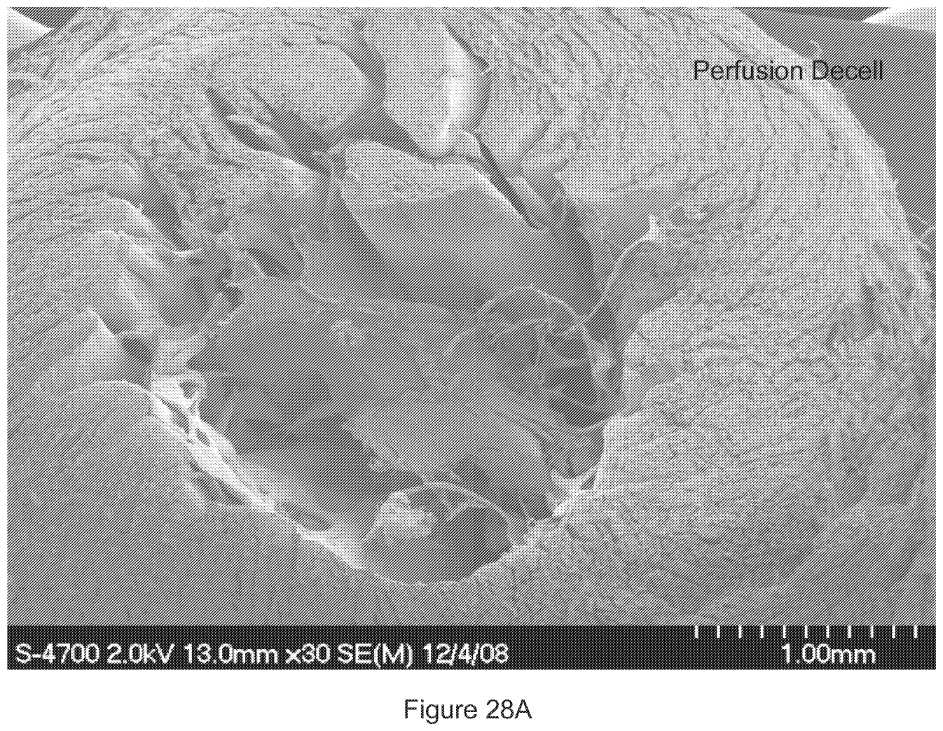

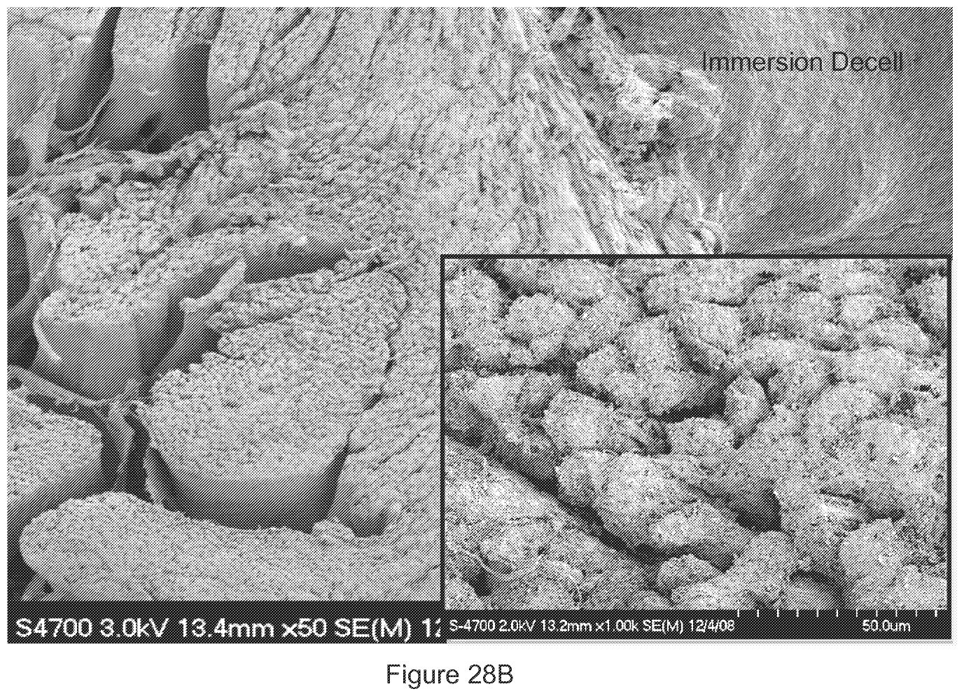

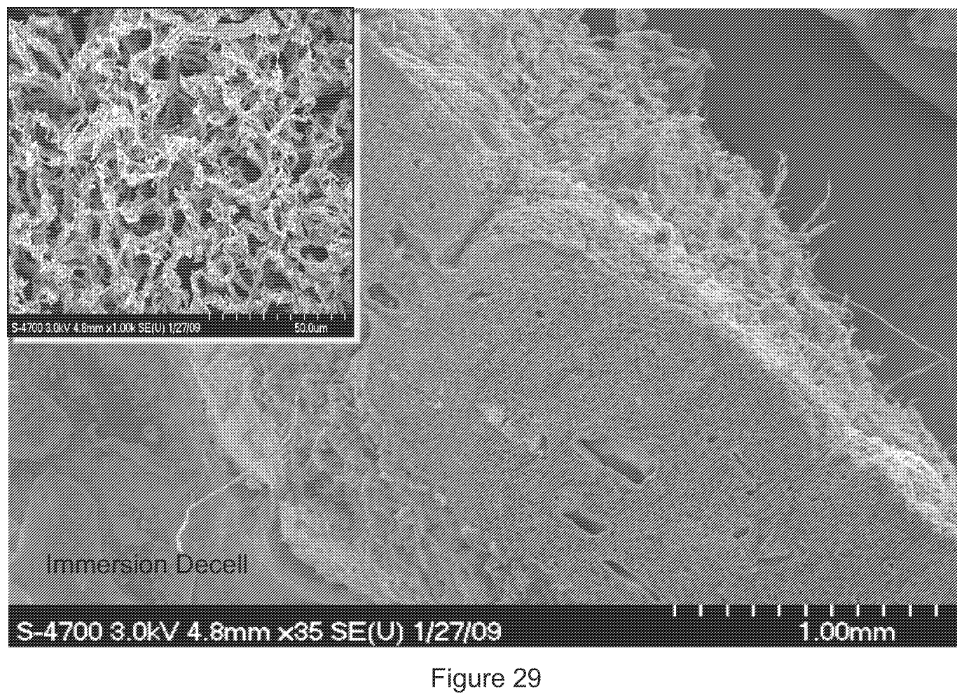

FIG. 27 shows SEM photographs of decellularized kidney. FIG. 27A shows a perfusion-decellularized kidney, while FIG. 27B shows an immersion-decellularized kidney. FIG. 28A shows a SEM photograph of a perfusion-decellularized heart, while FIG. 28B shows a SEM photograph of an immersion-decellularized heart. FIG. 29 shows a SEM photograph of an immersion-decellularized liver. These images further demonstrate the damage that immersion-decellularization caused to the ultrastructure of the organ, and the viability of the matrix following perfusion-decellularization.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of making a liver, comprising
providing a decellularized liver, wherein said decellularized liver comprises a decellularized extracellular matrix of said liver, wherein said extracellular matrix comprises an exterior surface, and wherein said extracellular matrix, including the vascular tree, substantially retains the morphology of said extracellular matrix prior to decellularization, and wherein said exterior surface is substantially intact; and
contacting said decellularized liver with about 40,000 or more regenerative cells under conditions in which said cells engraft, multiply and/or differentiate within and on said decellularized liver.

2. The method of claim 1, wherein said decellularized liver is contacted with about 23 million or more regenerative cells.

3. The method of claim 1, wherein said decellularized liver is contacted with about 30 million or more regenerative cells.

4. The method of claim 1, wherein said decellularized liver is contacted with about 35 million or more regenerative cells.

5. The method of claim 1, wherein said regenerative cells are hepatocytes.

6. The method of claim 1, wherein said regenerative cells are infused into said decellularized liver via a portal vein.

7. The method of claim 1, wherein said regenerative cells are injected into said decellularized liver.

8. A method of making a liver lobe, comprising
providing a decellularized liver or lobe-containing portion thereof, wherein said decellularized liver or lobe-containing portion thereof comprises a decellularized extracellular matrix of said liver or lobe-containing portion thereof, wherein said extracellular matrix comprises an exterior surface, and wherein said extracellular matrix, including the vascular tree, substantially retains the morphology of said extracellular matrix prior to decellularization, and wherein said exterior surface is substantially intact; and
contacting a lobe of said decellularized liver or lobe-containing portion thereof with a population of regenerative cells under conditions in which said regenerative cells engraft, multiply and/or differentiate within and on said decellularized liver lobe.

9. The method of claim 8, wherein said regenerative cells are primary hepatocytes.

10. The method of claim 8, wherein said regenerative cells are infused into said lobe via a portal vein.

11-15. (canceled)

16. A decellularized mammalian adrenal gland, comprising
a decellularized extracellular matrix of said adrenal gland, wherein said extracellular matrix comprises an exterior surface, and wherein said extracellular matrix, including the vascular tree, substantially retains the morphology of said extracellular matrix prior to decellularization, and wherein said exterior surface is substantially intact.
